(19) United States
(12) Patent Application Publication
(10) Pub. No.: US 2012/0071437 A1
(43) Pub. Date: Mar. 22, 2012

(54) TUNABLE CROSSLINKED POLYSACCHARIDE COMPOSITIONS

(75) Inventors: Dimitrios Stroumpoulis, Goleta, CA (US); Ahmet Tezel, Goleta, CA (US)

(73) Assignee: ALLERGAN, INC., Irvine, CA (US)

(21) Appl. No.: 13/206,454

(22) Filed: Aug. 9, 2011

Related U.S. Application Data
(63) Continuation-in-part of application No. 12/910,466, filed on Oct. 22, 2010.
Application No. 12/910,466 is a continuation-in-part of application No. 12/178,574, filed on Jul. 23, 2008.
(60) Provisional application No. 60/952,770, filed on Jul. 30, 2007.

(51) Int. Cl. A61K 31/715; A61P 17/00

(52) U.S. Cl. 514/54

(57) ABSTRACT

The present specification generally relates to injectable dermal fillers including multifunctional polyethylene glycol-based crosslinking agents, hydrogel compositions comprising a matrix polymer crosslinked with such crosslinking agents, and methods of treating a soft tissue condition using such hydrogel compositions.

### RELATED APPLICATIONS

This is a continuation-in-part of U.S. patent application Ser. No. 12/910,466, filed on Oct. 22, 2010, which is a continuation-in-part of U.S. patent application Ser. No. 12/178,574, filed on Jul. 30, 2008, which claims the benefit of and priority to U.S. Provisional Patent Application Ser. No. 60/952,770 filed on Jul. 30, 2007, the entire disclosure of each of these applications being incorporated herein by this specific reference.

### BACKGROUND

Hyaluronan, also known as hyaluronic acid (HA) is a non-sulfated glycosaminoglycan that is distributed widely throughout the human body in connective, epithelial, and neural tissues. Hyaluronan is abundant in the different layers of the skin, where it has multiple functions such as, e.g., to ensure good hydration, to assist in the organization of the extracellular matrix, to act as a filler material; and to participate in tissue repair mechanisms. However, with age, the quantity of hyaluronan, collagen, elastin, and other matrix polymers present in the skin decreases. For example, repeated exposed to ultra violet light, e.g., from the sun, causes dermal cells to both decrease their production of hyaluronan as well as increase the rate of its degradation. This hyaluronan loss results in various skin conditions such as, e.g., imperfects, defects, diseases and/or disorders, and the like.

Dermal fillers are useful in treating soft tissue condition and in other skin therapies because the fillers can replace lost endogenous matrix polymers, or enhance/facilitate the function of existing matrix polymers, in order to treat these skin conditions. In the past, such compositions have been used in cosmetic applications to fill wrinkles, lines, folds, scars, and to enhance dermal tissue, such as, e.g., to plump thin lips, or fill-in sunken eyes or shallow cheeks. One common matrix polymer used in dermal filler compositions is hyaluronan. Because hyaluronan is natural to the human body, it is a generally well tolerated and a fairly low risk treatment for a wide variety of skin conditions.

Originally, compositions comprising hyaluronan where made from naturally-occurring polymers, which exist in an uncrosslinked state. Although exhibiting excellent biocompatibility and affinity for water molecules, naturally-occurring hyaluronan exhibits poor biomechanical properties as a dermal filler. Tezel and Fredrickson, The Science of Hyaluronic Acid Dermal Fillers, J Cosmet Laser Ther. 10(1): 35-42 (2008); Kablik, et al., Comparative Physical Properties of Hyaluronic Acid Dermal Fillers, Dermatol Surg. 35 Suppl 1: 302-312 (2009); Beasley, et al., Hyaluronic Acid Fillers: A Comprehensive Review, Facial Plast Surg. 25(2): 86-94 (2009); each of which is hereby incorporated by reference in its entirety. One primary reason is that because this polymer is uncrosslinked, it is highly soluble and, as such, is cleared rapidly when administered into a skin region. Tezel, supra, 2008; Kablik, supra, 2009; Beasley, supra, 2009. This in vivo clearance is primarily achieved by rapid degradation of the polymers, principally enzymatic degradation via hyaluronidase and chemical degradation via free-radicals. Thus, while still in commercial use, compositions comprising uncrosslinked hyaluronan polymers tend to degrade within a few days after administration and thus require fairly frequent reinjection to maintain their skin improving effect.

To minimize the effect of these in vivo degradation pathways, matrix polymers are crosslinked to one another to form a stabilized hydrogel. Because hydrogels comprising crosslinked matrix polymers are a more solid substance, dermal fillers comprising such hydrogels remain in place at the implant site longer. Tezel, supra, 2008; Kablik, supra, 2009; Beasley, supra, 2009. In addition, these hydrogels are more suitable as a dermal filler because it's more solid nature improves the mechanical properties of the filler, allowing the filler to better lift and fill a skin region. Tezel, supra, 2008; Kablik, supra, 2009; Beasley, supra, 2009. Hyaluronan polymers are typically crosslinked with a bifunctional crosslinking agent, such as 1,4-butanediol diglycidyl ether, where a double ether bond links hyaluronan polymers to form a less water soluble hydrogel network that is more resistant to degradation, and thus requires less frequent reinjection, than the non-crosslinked hyaluronan compositions.

While known compositions comprising crosslinked hyaluronan last longer than their non-crosslinked counterparts, their duration is typically twelve months or less, thus still requiring fairly frequent reinjection. It is thus desirable to develop a hydrogel composition that is biocompatible and useful as a dermal filler, but has a longer useful lifetime upon injection. Specifically, it is desirable to develop a crosslinked hyaluronan composition that is biocompatible and injectable, but that has a higher mechanical strength, a greater resistance to enzymatic degradation, and a higher water retention than currently available compositions.

### BRIEF SUMMARY

Aspects of the present specification disclose multifunctional polyethylene glycol (PEG)-based crosslinking agents. A multifunctional PEG-based crosslinking agent may be a bifunctional PEG-based crosslinking agent, a trifunctional PEG-based crosslinking agent, a tetrafunctional PEG-based crosslinking agent, a pentafunctional PEG-based crosslinking agent, a hexafunctional PEG-based crosslinking agent, a heptafunctional PEG-based crosslinking agent, an octafunctional PEG-based crosslinking agent, a nonafunctional PEG-based crosslinking agent, or a decafunctional PEG-based crosslinking agent.

Other aspects of the present specification disclose hydrogel compositions comprising crosslinked matrix polymers, wherein the polymers are crosslinked with a multifunctional PEG-based crosslinking agent. A hydrogel composition comprising crosslinked polymers may be crosslinked with two or more different types of multifunctional PEG-based crosslinking agents. The PEG-based crosslinking agent may be any of the PEG-based crosslinking agents disclosed herein. The hydrogel composition may further comprise matrix polymers crosslinked with a non-PEG-based crosslinking agent. The non-PEG-based crosslinking agent may be divinyl sulfone (DVS), 1,4-butanediol diglycidyl ether (BDDE), 1,2-bis(2,3-epoxypropoxy)ethylene (EGDGE), 1,2,7,8-diepoxyoctane (DEO), biscarbodiimide (BCDI), adipic dihydrazide (ADH), bis(sulfosuccinimidyl)suberate (BS), hexamethylenediamine (NMDA), 1-(2,3-epoxypropyl)-2,3-epoxycyclohexane, or any combination thereof.

Other aspects of the present specification disclose methods of treating a soft tissue condition of an individual, the method comprising the step of administering a hydrogel composition disclosed herein to a site of the soft tissue condition, wherein administration of the composition eliminates a symptom of the soft tissue condition, or improves an aspect of the soft tissue condition, thereby treating the soft tissue site. A soft tissue condition includes a soft tissue imperfection, defect, disease, and/or disorder. Non-limiting examples of a soft tissue condition include a breast imperfection, defect, disease, and/or disorder, such as, e.g., breast augmentation, breast reconstruction, micromastia, thoracic hypoplasia, Poland's syndrome, defects due to implant complications like capsular contraction and/or rupture; facial imperfection, defect, disease, and/or disorder, such as, e.g., facial augmentation, facial reconstruction, Parry-Romberg syndrome, lupus erythematosus profundus, dermal divots, sunken cheeks, thin lips, retro-orbital defects, a facial fold, line and/or wrinkle like a glabellar line, a nasolabial line, a perioral line, and/or a marionette line, and/or other contour deformities or imperfections of the face; other soft tissue imperfections, defects, diseases, and/or disorders, such as, e.g., augmentation or reconstruction of the buttocks, calves, eye, genitals, and/or plantar fat pad and/or other contour deformities or imperfections of a body part, region or area; urinary incontinence, fecal incontinence, other forms of incontinence; and gastroesophageal reflux disease (GERD).

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a graph showing the difference in hardness, as measured by the elastic modulus G′, between a hydrogel composition comprising hyaluronan polymers crosslinked with BDDE, and a hydrogel composition comprising hyaluronan polymers crosslinked with a combination of BDDE and a tetrafunctional PEG-based crosslinker disclosed herein.

FIG. 2 is a graph showing the differences in hardness, as measured by the elastic modulus G′, and cohesivity, as measured by maximum compression force, between a hydrogel composition comprising hyaluronan polymers crosslinked with a tetrafunctional PEG-based crosslinker disclosed herein and two commercially available hyaluronan-based dermal fillers. Test sample 1 is a hyaluronan hydrogel crosslinked with a tetrafunctional PEG-based crosslinker disclosed herein; test sample 2 is JUVÉDERM® Ultra (Allergan, Inc., Irvine Calif.); and test sample 3 is PREVELLE™ Silk (Mentor Corp., Santa Barbara, Calif.).

FIG. 3 is a graph showing the force required, as measured by the extrusion force, to inject a hydrogel through a needle using a syringe. The hydrogels tested were a hydrogel composition comprising hyaluronan polymers crosslinked with a tetrafunctional PEG-based crosslinker disclosed herein and two commercially available hyaluronan-based dermal fillers. Test sample 1 is a hyaluronan hydrogel crosslinked with a tetrafunctional PEG-based crosslinker disclosed herein; test sample 2 is JUVÉDERM® Ultra (Allergan, Inc., Irvine Calif.); and test sample 3 is JUVÉDERM® Ultra Plus (Allergan, Inc., Irvine Calif.).

FIG. 4 shows the results of in vivo testing of a hydrogel composition comprising hyaluronan polymers crosslinked with a tetrafunctional PEG-based crosslinker disclosed herein and two commercially available hyaluronan-based dermal fillers. FIG. 4A is a graph showing the duration of the three different hydrogel samples implanted into the shoulder area of rats. FIG. 4B is a graph showing the shape retention characteristic of the three different hydrogel samples implanted into the shoulder area of rats. FIG. 4C is a graph showing the duration of the three different hydrogel samples implanted into the hip area of rats. FIG. 4D is a graph showing the shape retention characteristic of the three different hydrogel samples implanted into the hip area of rats. For all graphs, test sample 1 is a hyaluronan comprising crosslinked with a tetrafunctional PEG-based crosslinker disclosed herein; test sample 2 is JUVÉDERM® Ultra (Allergan, Inc., Irvine Calif.); and test sample 3 is PREVELLE™ Silk (Mentor Corp., Santa Barbara, Calif.).

### DETAILED DESCRIPTION

The present disclosure generally relates to novel multifunctional PEG-based crosslinking agents, hydrogel compositions comprising glycosaminoglycan polymers crosslinked using such PEG-based crosslinking agents, and methods of treating a soft tissue condition using such compositions. Because the structural configuration of PEG can be widely varied based on, e.g., the length of a PEG polymer chain, the degree of polymer chain branching, and any combination thereof, a PEG-based crosslinker can be tuned based on the desired properties of the crosslinked glycosaminoglycan. The multifunctional PEG-based crosslinking agents disclosed herein are able to link more polymer strands of glycosaminoglycans when compared to traditional bifunctional crosslinking agents. As such, the multifunctional PEG-based crosslinkers disclosed herein results in crosslinked glycosaminoglycan compositions with greater mechanical strength (G′) and improved resistance to degradation. Moreover, the PEG-based crosslinkers disclosed herein increase the probability of each molecule of a crosslinking agent reacting with at least one glycosaminoglycan polymer strand, thereby facilitating purification and removal of unreacted crosslinking agents from the final hydrogel composition.

Aspects of the present specification disclose, in part, a polyethylene glycol (PEG)-based crosslinking agent. As used herein, the term “PEG-based crosslinking agent” is synonymous with “PEG-based crosslinker” and refers to a PEG molecule comprising at least two reactive sites useful to covalently conjugate another molecule to the PEG molecule. PEG comprises a group of biocompatible, hydrophilic, and inert polymers having the general formula HO(CH₂CH₂O)ₙH, where n is an integer from 2 to 100, which is synthesized by the polymerization of ethylene oxide. A PEG molecule can be linear or branched. Branched PEGs include, without limitation, forked PEGs, star PEGs, comb PEGs, brush PEGs, and graft PEGs. A forked PEG is a branched PEG comprising two polymer chains emanating from a single branch point. A star PEG is a branched PEG comprising three or more linear polymer chains emanating from a central core group or a single branch point. A comb PEG is a branched PEG comprising two or more three-way branch points and linear side chains emanating from a main backbone polymer chain. A brush PEG is a branched PEG comprising three or more linear polymer chains emanating from a main backbone polymer chain. A graft PEG is a branched PEG comprising two or more polymer chains where one or more polymer chains are different, structurally or configurationally, from the main chain. The polymer chains comprising a PEG may be blocked.

In standard nomenclature, a branched PEG can be referred to by the number of polymer chains is comprises. Thus, a branched PEG having three polymer chains is referred to as a three-arm PEG or 3-arm PEG, a branched PEG having four polymer chains is referred to as a four-arm PEG or 4-arm PEG, a branched PEG having five polymer chains is referred to as a five-arm PEG or 5-arm PEG, a branched PEG having six polymer chains is referred to as a six-arm PEG or 6-arm PEG, a branched PEG having seven polymer chains is referred to as a seven-arm PEG or 7-arm PEG, etc. The physical properties of PEG, such as melting point, cohesiveness, and viscosity, can be altered by varying the length of the polymer chain, the type of initiator used during the polymerization process, and/or whether the PEG has a linear or branched configuration. PEG molecules, both linear and branched, are commercially available over a wide range of molecular weights from 300 g/mol to 10,000,000 g/mol.

A polymer chain of a PEG-based crosslinking agent may be functional in that it comprises a reactive site used to conjugate the PEG chain to another molecule. A PEG-based crosslinker containing more than one reactive site is referred to generally as a multifunctional PEG-based crosslinker, or more specifically by the number of reactive sites it contains. For example, a bifunctional PEG-based crosslinker has two reactive sites useful for crosslinking purposes, a trifunctional PEG-based crosslinker has three reactive sites useful for crosslinking purposes, a tetrafunctional PEG-based crosslinker has four reactive sites useful for crosslinking purposes, a pentafunctional PEG-based crosslinker has five reactive sites useful for crosslinking purposes, a hexafunctional PEG-based crosslinker has six reactive sites useful for crosslinking purposes, a heptafunctional PEG-based crosslinker has seven reactive sites useful for crosslinking purposes, etc. The number of functional sites on a PEG-based crosslinker disclosed herein is limited only by the ability of the hyaluronic acid polymer strands to bind to the resulting active sites on the crosslinker due to, e.g., geometry and steric hindrance.

A polymer chain of a PEG-based crosslinking agent is made functional by attaching a reactive group to the free end of a polymer chain from a base PEG molecule. Any reactive group that can be used to covalently join glycosaminoglycan polymers to the PEG-based crosslinker may be used, including, without limitation, epoxides. The PEG-based crosslinking agents disclosed herein may be made according to any PEG synthesis methods known to one of ordinary skill in the art. Generally, a multifunctional PEG-based crosslinking agent is synthesized from a base poly-alcohol or PEG molecule having the desired chain length and branching by attaching epoxide groups. Such epoxide groups can be attached to the base poly-alcohol or PEG molecule by deprotonating the hydroxyl groups and reacting with epichlorohydrin. Example 1 describes the synthesis of a specific PEG-based crosslinking agent disclosed herein.

A PEG-based crosslinker may have a variety of polymer chain lengths which affect its mechanical properties. As such, a PEG-based crosslinking agent disclosed herein is of tunable size. In an aspect of this embodiment, a trifunctional PEG-based crosslinking agent comprises three polymer chains emanating from a central core group, with each chain having the structure CH₂(OCH₂CH₂)ₙOCH₂epoxide, where n is an integer from 0 to 60. In another aspect of this embodiment, a tetrafunctional PEG-based crosslinking agent comprises four polymer chains emanating from a central core group with each chain having the structure CH₂(OCH₂CH₂)ₙOCH₂epoxide, where n is an integer from 0 to 60. In yet another aspect of this embodiment, a pentafunctional PEG-based crosslinking agent comprises five polymer chains emanating from a central core group, with each chain having the structure CH₂(OCH₂CH₂)ₙOCH₂epoxide, where n is an integer from 0 to 60. In still another aspect of this embodiment, a hexafunctional PEG-based crosslinking agent comprises six polymer chains emanating from a central core group, with each chain having the structure CH₂(OCH₂CH₂)ₙOCH₂epoxide, where n is an integer from 0 to 60. In a further aspect of this embodiment, a heptafunctional PEG-based crosslinking agent comprises seven polymer chains emanating from a central core group, with each chain having the structure CH₂(OCH₂CH₂)ₙOCH₂epoxide, where n is an integer from 0 to 60. In a yet further aspect of this embodiment, an octafunctional PEG-based crosslinking agent comprises eight polymer chains emanating from a central core group, with each chain having the structure CH₂(OCH₂CH₂)ₙOCH₂epoxide, where n is an integer from 0 to 60. In a still further aspect of this embodiment, a nonafunctional PEG-based crosslinking agent comprises nine polymer chains emanating from a central core group, with each chain having the structure CH₂(OCH₂CH₂)ₙOCH₂epoxide, where n is an integer from 0 to 60. In another aspect of this embodiment, a decafunctional PEG-based crosslinking agent comprises ten polymer chains emanating from a central core group, with each chain having the structure CH₂(OCH₂CH₂)ₙOCH₂epoxide, where n is an integer from 0 to 60. A core group can be a carbon atom, a generational carbon like a first or second generation carbon, or a dendrite.

In another aspect of this embodiment, a trifunctional PEG-based crosslinking agent comprises three polymer chains emanating from a PEG polymer backbone having the structure HO(CH₂CH₂O)ₘH, where m is an integer from 2 to 100, with each chain having the structure CH₂(OCH₂CH₂)ₙOCH₂epoxide, where n is an integer from 0 to 60. In another aspect of this embodiment, a tetrafunctional PEG-based crosslinking agent comprises four polymer chains emanating from a PEG polymer backbone having the structure HO(CH₂CH₂O)ₘH, where m is an integer from 2 to 100, with each chain having the structure CH₂(OCH₂CH₂)ₙOCH₂epoxide, where n is an integer from 0 to 60. In yet another aspect of this embodiment, a pentafunctional PEG-based crosslinking agent comprises five polymer chains emanating from a PEG polymer backbone having the structure HO(CH₂CH₂O)ₘH, where m is an integer from 2 to 100, with each chain having the structure CH₂(OCH₂CH₂)ₙOCH₂epoxide, where n is an integer from 0 to 60. In still another aspect of this embodiment, a hexafunctional PEG-based crosslinking agent comprises six polymer chains emanating from a PEG polymer backbone having the structure HO(CH₂CH₂O)ₘH, where m is an integer from 2 to 100, with each chain having the structure CH₂(OCH₂CH₂)ₙOCH₂epoxide, where n is an integer from 0 to 60. In a further aspect of this embodiment, a heptafunctional PEG-based crosslinking agent comprises seven polymer chains emanating from a PEG polymer backbone having the structure HO(CH₂CH₂O)ₘH, where m is an integer from 0 to 100, with each chain having the structure CH₂(OCH₂CH₂)ₙOCH₂epoxide, where n is an integer from 0 to 60. In a yet further aspect of this embodiment, an octafunctional PEG-based crosslinking agent comprises eight polymer chains emanating from a PEG polymer backbone having the structure HO(CH₂CH₂O)ₘH, where m is an integer from 2 to 100, with each chain having the structure CH₂(OCH₂CH₂)ₙOCH₂epoxide, where n is an integer from 0 to 60. In a still further aspect of this embodiment, a nonafunctional PEG-based crosslinking agent comprises nine polymer chains emanating from a PEG polymer backbone having the structure HO(CH₂CH₂O)ₘH, where m is an integer from 2 to 100, with each chain having the structure CH₂(OCH₂CH₂)ₙOCH₂epoxide, where n is an integer from 0 to 60. In another aspect of this embodiment, a decafunctional PEG-based crosslinking agent comprises ten polymer chains emanating from a PEG polymer backbone having the structure HO(CH₂CH₂O)ₘH, where m is an integer from 2 to 100, with each chain having the structure CH₂(OCH₂CH₂)ₙOCH₂epoxide, where n is an integer from 0 to 60.

In another aspect of this embodiment, a bifunctional PEG-based crosslinking agent has a molecular weight of between about 200 Da to about 10,000 Da. In yet another aspect of this embodiment, a trifunctional PEG-based crosslinking agent has a molecular weight of between about 200 Da to about 10,000 Da. In still another aspect of this embodiment, a tetrafunctional PEG-based crosslinking agent has a molecular weight of between about 200 Da to about 10,000 Da. In a further aspect of this embodiment, a pentafunctional PEG-based crosslinking agent has a molecular weight of between about 200 Da to about 10,000 Da. In another aspect of this embodiment, a hexafunctional PEG-based crosslinking agent has a molecular weight of between about 200 Da to about 10,000 Da.

Matrix polymers, such as e.g., polysaccharides polymers like glycosaminoglycan polymers, may be crosslinked with only one type of multifunctional PEG-based crosslinker or with two or more different types of multifunctional PEG-based crosslinkers. In an aspect of this embodiment, glycosaminoglycan polymer strands may be crosslinked solely with a trifunctional PEG-based crosslinker, a tetrafunctional PEG-based crosslinker, a pentafunctional PEG-based crosslinker, a hexafunctional PEG-based crosslinker, a heptafunctional PEG-based crosslinker, an octafunctional PEG-based crosslinker, a nonafunctional PEG-based crosslinker, or a decafunctional PEG-based crosslinker. In other aspects of this embodiment, glycosaminoglycan polymer strands may be crosslinked using a combination of, e.g., trifunctional and tetrafunctional PEG-based crosslinkers, trifunctional and pentafunctional PEG-based crosslinkers, tetrafunctional and pentafunctional PEG-based crosslinkers, tetrafunctional and hexafunctional PEG-based crosslinkers, tetrafunctional and octafunctional PEG-based crosslinkers, pentafunctional and hexafunctional PEG-based crosslinkers, pentafunctional and hepafunctional PEG-based crosslinkers, or pentafunctional and nonafunctional PEG-based crosslinkers. By selecting the multifuntionality of the PEG-based crosslinkers and/or varying the amounts of the different types of multifunctional PEG-based crosslinkers, the mechanical strength of the resulting hydrogel can be tailored to the desired specifications (see, e.g., Examples 5, 6, and 7).

Matrix polymers, such as e.g., polysaccharides polymers like glycosaminoglycan polymers, may be crosslinked solely with the multifunctional PEG-based crosslinkers disclosed herein or in combination with any other crosslinking agent suitable for making crosslinked hyaluronan. Non limiting examples of such crosslinking agents include dialdehydes and disufides crosslinking agents including, without limitation, divinyl sulfones, diglycidyl ethers, and bis-epoxides. Non-limiting examples of hyaluronan crosslinking agents include divinyl sulfone (DVS), 1,4-butanediol diglycidyl ether (BDDE), 1,2-bis(2,3-epoxypropoxy)ethylene (EGDGE), 1,2,7,8-diepoxyoctane (DEO), biscarbodiimide (BCDI), adipic dihydrazide (ADH), bis(sulfosuccinimidyl)suberate (BS), hexamethylenediamine (NMDA), 1-(2,3-epoxypropyl)-2,3-epoxycyclohexane, or combinations thereof. By mixing a PEG-based crosslinker disclosed herein with another crosslinker, such as, e.g., the ones disclosed herein, in varying ratios, the mechanical strength and hardness of the final hyaluronan composition may be tuned as desired (see, e.g., Examples 5, 6, and 7).

In one aspect of this embodiment, glycosaminoglycan polymers are crosslinked using a combination of PEG-based crosslinkers disclosed herein and BDDE. In another aspect of this embodiment, glycosaminoglycan polymer strands are crosslinked using a combination of PEG-based crosslinkers disclosed herein and EGDGE. In yet another aspect of this embodiment, glycosaminoglycan polymer strands are crosslinked using a combination of PEG-based crosslinkers disclosed herein and DEO. In still another aspect of this embodiment, glycosaminoglycan polymer strands are crosslinked using a combination of PEG-based crosslinkers disclosed herein and DVS.

Matrix polymers, such as e.g., polysaccharides polymers like glycosaminoglycan polymers, are crosslinked using the PEG-based crosslinking agents disclosed herein using conventional procedures known to a person of ordinary skill. For example, glycosaminoglycan polymers are brought into contact with a PEG-based crosslinker and allowed to react. The glycosaminoglycan polymers may be reacted with more than one PEG-based crosslinker as disclosed herein in either a step-wise fashion, with a lower functionality PEG-based crosslinker being brought into contact first or with a higher functionality PEG-based crosslinker being brought into contact first. Alternatively, glycosaminoglycan polymers may be reacted with a plurality of PEG-based crosslinkers in one step.

Matrix polymers, such as e.g., polysaccharides polymers like glycosaminoglycan polymers, that may be crosslinked using the PEG-based crosslinking agents and methods disclosed herein. Additional matrix polymers, such as e.g., polysaccharides polymers like glycosaminoglycan polymers, that may be crosslinked using the PEG-based crosslinking agents and methods disclosed herein are described in, e.g., Piron and Tholin, Polysaccharide Crosslinking, Hydrogel Preparation, Resulting Polysaccharides(s) and Hydrogel(s), uses Thereof, U.S. Patent Publication 2003/0148995; Lebreton, Cross-Linking of Low and High Molecular Weight Polysaccharides Preparation of Injectable Monophase Hydrogels and Polysaccharides and Hydrogels thus Obtained, U.S. Patent Publication 2006/0194758; Lebreton, Viscoelastic Solutions Containing Sodium Hyaluronate and Hydroxypropyl Methyl Cellulose, Preparation and Uses, U.S. Patent Publication 2008/0089918; Stroumpoulis, et al., Polysaccharide Gel Formulations Having Increased Longevity, U.S. Patent Publication 2009/014333; Stroumpoulis, et al., Polysaccharide Gel Formulations Having Increased Longevity, U.S. Patent Publication 2010/0004198; Lebreton, Hyaluronic Acid-Based Gels Including Lidocaine, U.S. Patent Publication 2010/0028438; Stroumpoulis, et al., Polysaccharide Gel Formulations Having Multistage Bioactive Agent Delivery, U.S. Patent Publication 2010/0098764; and Di Napoli, Composition and Method for Intradermal Soft Tissue Augmentation, International Patent Publication WO 2004/073759, each of which is hereby incorporated by reference in its entirety.

Any conventional crosslinking method may be used to crosslink glycosaminoglycan polymers using a multifunctional PEG-based crosslinker disclosed herein alone, with another type of multifunctional PEG-based crosslinker, and/or with conjunction with a non-PEG-based crosslinker. Generally, a matrix polymer undergoes a preparation step and then is simply mixed with a crosslinker in order to initiate the crosslinking reaction. For example, a glycosaminoglycan is first hydrated by mixing the polymer with a 0.01-1% sodium hydroxide solution and incubating at ambient temperature for about 1 hour to about 5 hours. Next, about 10% (w/w) to about 25% (w/w), or about 50 mg to about 2,000 mg, of an appropriate multifunctional PEG-based crosslinking agent(s) (about 200 Da to about 10,000 Da) is added to the hydrated glycosaminoglycan. If a non-PEG-based crosslinker is also employed, about 20 to about 200 mg of non-PEG-based crosslinker is added as well. The mixture is then mechanically homogenized, and then placed in an about 40 to about 70° C. oven for about 1 hour to about 10 hours. The resulting crosslinked hydrogel is neutralized with an equimolar amount of hydrochloric acid and swelled in a physiologically-acceptable solution, such as, e.g., a buffered solution of about pH 5.5 to about pH 8.5.

In one aspect of this embodiment, a crosslinking reaction comprises about 90% (w/w) glycosaminoglycan polymer and about 10% multifunctional PEG-based crosslinking agent, a crosslinking reaction comprises about 89% (w/w) glycosaminoglycan polymer and about 11% multifunctional PEG-based crosslinking agent, a crosslinking reaction comprises about 88% (w/w) glycosaminoglycan polymer and about 12% multifunctional PEG-based crosslinking agent, a crosslinking reaction comprises about 87% (w/w) glycosaminoglycan polymer and about 13% multifunctional PEG-based crosslinking agent, a crosslinking reaction comprises about 86% (w/w) glycosaminoglycan polymer and about 14% multifunctional PEG-based crosslinking agent, a crosslinking reaction comprises about 85% (w/w) glycosaminoglycan polymer and about 15% multifunctional PEG-based crosslinking agent, a crosslinking reaction comprises about 84% (w/w) glycosaminoglycan polymer and about 16% multifunctional PEG-based crosslinking agent, a crosslinking reaction comprises about 83% (w/w) glycosaminoglycan polymer and about 17% multifunctional PEG-based crosslinking agent, a crosslinking reaction comprises about 82% (w/w) glycosaminoglycan polymer and about 18% multifunctional PEG-based crosslinking agent, a crosslinking reaction comprises about 81% (w/w) glycosaminoglycan polymer and about 19% multifunctional PEG-based crosslinking agent, or a crosslinking reaction comprises about 80% (w/w) glycosaminoglycan polymer and about 20% multifunctional PEG-based crosslinking agent.

In another aspect of this embodiment, a crosslinking reaction comprises about 90% (w/w) glycosaminoglycan polymer and about 10% pentaerythritol tetraglycidyl ether (PEGE) crosslinking agent, a crosslinking reaction comprises about 89% (w/w) glycosaminoglycan polymer and about 11% PEGE crosslinking agent, a crosslinking reaction comprises about 88% (w/w) glycosaminoglycan polymer and about 12% PEGE, a crosslinking reaction comprises about 87% (w/w) glycosaminoglycan polymer and about 13% PEGE, a crosslinking reaction comprises about 86% (w/w) glycosaminoglycan polymer and about 14% PEGE, a crosslinking reaction comprises about 85% (w/w) glycosaminoglycan polymer and about 15% PEGE, a crosslinking reaction comprises about 84% (w/w) glycosaminoglycan polymer and about 16% PEGE, a crosslinking reaction comprises about 83% (w/w) glycosaminoglycan polymer and about 17% multifunctional PEGE, a crosslinking reaction comprises about 82% (w/w) glycosaminoglycan polymer and about 18% PEGE, a crosslinking reaction comprises about 81% (w/w) glycosaminoglycan polymer and about 19% PEGE, or a crosslinking reaction comprises about 80% (w/w) glycosaminoglycan polymer and about 20% PEGE.

In a specific embodiment of the invention, the hyaluronan polymer has a mean molecular weight of between about 310,000 Da and about 840,000 Da and is crosslinked with PEGE. The initial PEGE crosslinking reaction is about 10% to about 15% (w/w), for example, about 13% PEGE (w/w). The concentration of NaHA in the final composition is about 20 to about 30 mg HA/g (mg/g).

Aspects of the present specification provide, in part, a hydrogel composition comprising glycosaminoglycan polymers. As used herein, the term “glycosaminoglycan” is synonymous with “GAG” and “mucopolysaccharide” and refers to long unbranched polysaccharides consisting of a repeating disaccharide units. The repeating unit consists of a hexose (six-carbon sugar) or a hexuronic acid, linked to a hexosamine (six-carbon sugar containing nitrogen) and pharmaceutically acceptable salts thereof. Members of the GAG family vary in the type of hexosamine, hexose or hexuronic acid unit they contain, such as, e.g., glucuronic acid, iduronic acid, galactose, galactosamine, glucosamine) and may also vary in the geometry of the glycosidic linkage. Any glycosaminoglycan is useful in the compositions disclosed herein with the proviso that the glycosaminoglycan improves a soft tissue condition as disclosed herein. GAGs useful in the compositions and methods disclosed herein are commercially available. Table 1 lists representative GAGs.

TABLE 1
Examples of GAGs

| | | | Glycosidic | |
|---|---|---|---|---|
| | Hexuronic | | linkage | |
| Name | acid/Hexose | Hexosamine | geometry | Unique features |
| Chondroitin | GlcUA or | GalNAc or | -4GlcUAβ1- | Most prevalent GAG |
| sulfate | GlcUA(2S) | GalNAc(4S) or | 3GalNAcβ1- | |
| | | GalNAc(6S) or | | |
| | | GalNAc(4S,6S) | | |
| Dermatan | GlcUA or | GalNAc or | -4IdoUAβ1- | Distinguished from chondroitin |
| sulfate | IdoUA or | GalNAc(4S) or | 3GalNAcβ1- | sulfate by the presence of iduronic |
| | IdoUA(2S) | GalNAc(6S) or | | acid, although some hexuronic |
| | | GalNAc(4S,6S) | | acid monosaccharides may be |
| | | | | glucuronic acid. |
| Keratan | Gal or | GlcNAc or | -3Gal(6S)β1- | Keratan sulfate type II may be |
| sulfate | Gal(6S) | GlcNAc(6S) | 4GlcNAc(6S)β1- | fucosylated. |
| Heparin | GlcUA or | GlcNAc or | -4IdoUA(2S)α1- | Highest negative charge density of |
| | IdoUA(2S) | GlcNS or | 4GlcNS(6S)α1- | any known biological molecule |
| | | GlcNAc(6S) or | | |
| | | GlcNS(6S) | | |
| Heparan | GlcUA or | GlcNAc or | -4GlcUAβ1- | Highly similar in structure to |
| sulfate | IdoUA or | GlcNS or | 4GlcNAcα1- | heparin, however heparan sulfates |
| | IdoUA(2S) | GlcNAc(6S) or | | disaccharide units are organised |
| | | GlcNS(6S) | | into distinct sulfated and non- |
| | | | | sulfated domains. |
| Hyaluronan | GlcUA | GlcNAc | -4GlcUAβ1- | The only GAG that is exclusively |
| | | | 3GlcNAcβ1- | non-sulfated |

GlcUA = β-D-glucuronic acid
GlcUA(2S) = 2-O-sulfo-β-D-glucuronic acid
IdoUA = α-L-iduronic acid
IdoUA(2S) = 2-O-sulfo-α-L-iduronic acid
Gal = β-D-galactose
Gal(6S) = 6-O-sulfo-β-D-galactose
GalNAc = β-D-N-acetylgalactosamine
GalNAc(4S) = β-D-N-acetylgalactosamine-4-O-sulfate
GalNAc(6S) = β-D-N-acetylgalactosamine-6-O-sulfate
GalNAc(4S,6S) = β-D-N-acetylgalactosamine-4-O, 6-O-sulfate
GlcNAc = α-D-N-acetylglucosamine
GlcNS = α-D-N-sulfoglucosamine
GlcNS(6S) = α-D-N-sulfoglucosamine-6-O-sulfate

Aspects of the present specification provide, in part, a hydrogel composition comprising a chondroitin sulfate. As used herein, the term “chondroitin sulfate” refers to an unbranched, sulfated GAG of variable length comprising disaccharides of two alternating monosaccharides of D-glucuronic acid (GlcA) and N-acetyl-D-galactosamine (GalNAc) and pharmaceutically acceptable salts thereof. A chondroitin sulfate may also include D-glucuronic acid residues that are epimerized into L-iduronic acid (IdoA), in which case the resulting disaccharide is referred to as dermatan sulfate. A chondroitin sulfate polymer can have a chain of over 100 individual sugars, each of which can be sulfated in variable positions and quantities. Chondroitin sulfate is an important structural component of cartilage and provides much of its resistance to compression. Any chondroitin sulfate is useful in the compositions disclosed herein with the proviso that the chondroitin sulfate improves a soft tissue condition as disclosed herein. Non-limiting examples of pharmaceutically acceptable salts of chondroitin sulfate include sodium chondroitin sulfate, potassium chondroitin sulfate, magnesium chondroitin sulfate, calcium chondroitin sulfate, and combinations thereof.

Aspects of the present specification provide, in part, a hydrogel composition comprising a keratan sulfate. As used herein, the term “keratan sulfate” refers to a GAG of variable length comprising disaccharide units, which themselves include β-D-galactose and N-acetyl-D-galactosamine (GalNAc) and pharmaceutically acceptable salts thereof. Disaccharides within the repeating region of keratan sulfate may be fucosylated and N-acetylneuraminic acid caps the end of the chains. Any keratan sulfate is useful in the compositions disclosed herein with the proviso that the keratan sulfate improves a soft tissue condition as disclosed herein. Non-limiting examples of pharmaceutically acceptable salts of keratan sulfate include sodium keratan sulfate, potassium keratan sulfate, magnesium keratan sulfate, calcium keratan sulfate, and combinations thereof.

Aspects of the present specification provide, in part, a hydrogel composition comprising a hyaluronan. As used herein, the term “hyaluronan” is synonymous with “hyaluronic acid”, “HA”, “hyaluronic acid” and “hyaluronate”. Hyaluronan includes anionic, non-sulfated glycosaminoglycan polymers comprising disaccharide units, which themselves include D-glucuronic acid and D-N-acetylglucosamine monomers, linked together via alternating β-1,4 and β-1,3 glycosidic bonds and pharmaceutically acceptable salts thereof. Hyaluronan can be purified from animal and non-animal sources. Polymers of hyaluronan can range in size from about 5,000 Da to about 20,000,000 Da. Any hyaluronan is useful in the compositions disclosed herein with the proviso that the hyaluronan improves a soft tissue condition as disclosed herein. Non-limiting examples of pharmaceutically acceptable salts of hyaluronan include sodium hyaluronate, potassium hyaluronate, magnesium hyaluronate, calcium hyaluronate, and combinations thereof.

Aspects of the present specification provide, in part, a hydrogel composition comprising crosslinked glycosaminoglycan polymers. As used herein, the term “crosslinked” refers to the intermolecular bonds joining individual polymer molecules, or monomer chains, into a more stable structure like a gel. As such, a crosslinked glycosaminoglycan polymer has at least one intermolecular bond joining at least one individual glycosaminoglycan polymer molecule to another one. Any of the glycosaminoglycan polymers disclosed herein may be crosslinked. The amount of crosslinked glycosaminoglycan polymers present in a hydrogel composition depends of the properties and characteristic desired.

In an aspect of this embodiment, a hydrogel composition comprises crosslinked chondroitin sulfate polymers, crosslinked dermatan sulfate polymers, crosslinked keratan sulfate polymers, crosslinked heparan polymers, crosslinked heparan sulfate polymers, or crosslinked hyaluronan polymers.

In other aspects of this embodiment, a hydrogel composition comprises crosslinked glycosaminoglycan polymers where the crosslinked glycosaminoglycan polymers represents, e.g., about 1% by weight, about 2% by weight, about 3% by weight, about 4% by weight, about 5% by weight, about 6% by weight, about 7% by weight, about 8% by weight, or about 9%, or about 10% by weight, of the total amount of glycosaminoglycan polymers present in the composition. In yet other aspects of this embodiment, a hydrogel composition comprises crosslinked glycosaminoglycan polymers where the crosslinked glycosaminoglycan polymers represents, e.g., at most 1% by weight, at most 2% by weight, at most 3% by weight, at most 4% by weight, at most 5% by weight, at most 6% by weight, at most 7% by weight, at most 8% by weight, at most 9% by weight, or at most 10% by weight, of the total amount of glycosaminoglycan polymers present in the composition. In still other aspects of this embodiment, a hydrogel composition comprises crosslinked glycosaminoglycan polymers where the crosslinked glycosaminoglycan polymers represents, e.g., about 0% to about 10% by weight, about 1% to about 10% by weight, about 3% to about 10% by weight, or about 5% to about 10% by weight, of the total amount of glycosaminoglycan polymers present in the composition.

In other aspects of this embodiment, a hydrogel composition comprises crosslinked glycosaminoglycan polymers where the crosslinked glycosaminoglycan polymers represents, e.g., about 50% by weight, about 55% by weight, about 60% by weight, about 65% by weight, about 70% by weight, about 75% by weight, about 80% by weight, or about 85%, of the total amount of glycosaminoglycan polymers present in the composition. In yet other aspects of this embodiment, a hydrogel composition comprises crosslinked glycosaminoglycan polymers where the crosslinked glycosaminoglycan polymers represents, e.g., at least 50% by weight, at least 55% by weight, at least 60% by weight, at least 65% by weight, at least 70% by weight, at least 75% by weight, at least 80% by weight, or at least 85%, of the total amount of glycosaminoglycan polymers present in the composition. In still other aspects of this embodiment, a hydrogel composition comprises crosslinked glycosaminoglycan polymers where the crosslinked glycosaminoglycan polymers represents, e.g., about 50% to about 90% by weight, about 55% to about 85% by weight, about 50% to about 70% by weight, about 60% to about 80% by weight, about 70% to about 90% by weight, about 50% to about 60% by weight, about 60% to about 70% by weight, about 70% to about 80% by weight, about 80% to about 90% by weight, of the total amount of glycosaminoglycan polymers present in the composition.

In other aspects of this embodiment, a hydrogel composition comprises crosslinked glycosaminoglycan polymers where the crosslinked glycosaminoglycan polymers represents, e.g., about 90% by weight, about 91% by weight, about 92% by weight, about 93% by weight, about 94% by weight, about 95% by weight, about 96% by weight, about 97% by weight, about 98% by weight, about 99% by weight, or about 100% by weight, of the total amount of glycosaminoglycan polymers present in the composition. In yet other aspects of this embodiment, a hydrogel composition comprises crosslinked glycosaminoglycan polymers where the crosslinked glycosaminoglycan polymers represents, e.g., at least 90% by weight, at least 91% by weight, at least 92% by weight, at least 93% by weight, at least 94% by weight, at least 95% by weight, at least 96% by weight, at least 97% by weight, at least 98% by weight, at least 99% by weight, or at least 100% by weight, of the total amount of glycosaminoglycan polymers present in the composition. In still other aspects of this embodiment, a hydrogel composition comprises crosslinked glycosaminoglycan polymers where the crosslinked glycosaminoglycan polymers represents, e.g., about 90% to about 100% by weight, about 91% to about 100% by weight, about 93% to about 100% by weight, about 95% to about 100% by weight, or about 98% to about 100% by weight, of the total amount of glycosaminoglycan polymers present in the composition.

Aspects of the present specification provide, in part, a hydrogel composition comprising crosslinked glycosaminoglycan polymers having a degree of crosslinking. As used herein, the term “degree of crosslinking” refers to the percentage of monomeric units of a glycosaminoglycan polymer that are bound to a cross-linking agent, such as, e.g., the disaccharide monomer units of hyaluronan. Thus, a hydrogel composition comprising crosslinked glycosaminoglycan polymers with a 4% degree of crosslinking means that on average there are four crosslinking molecules for every 100 monomeric units. Every other parameter being equal, the greater the degree of crosslinking, the harder a composition comprising crosslinked glycosaminoglycan polymers becomes.

In other aspects of this embodiment, a hydrogel composition comprises crosslinked glycosaminoglycan polymers where the degree of crosslinking is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%. In yet other aspects of this embodiment, a hydrogel composition comprises crosslinked glycosaminoglycan polymers where the degree of crosslinking is at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, or at most 20%. In still other aspects of this embodiment, a hydrogel composition comprises crosslinked glycosaminoglycan polymers where the degree of crosslinking is about 1% to about 20%, about 2% to about 19%, about 3% to about 18%, about 4% to about 17%, about 5% to about 16%, about 1% to about 15%, about 2% to about 11%, about 3% to about 10%, about 1% to about 10%, about 1% to about 5%, about 5% to about 10%, about 10% to about 15%, about 11% to about 15%, about 6% to about 10%, or about 6% to about 8%.

Aspects of the present specification provide, in part, a hydrogel composition comprising uncrosslinked glycosaminoglycan polymers. As used herein, the term “uncrosslinked” refers to a lack of intermolecular bonds joining the individual matrix polymer molecules, or monomer chains. As such, an uncrosslinked glycosaminoglycan polymer is not linked to any other glycosaminoglycan polymers by an intermolecular bond.

In an embodiment, a hydrogel composition comprises uncrosslinked glycosaminoglycan polymers. In aspects of this embodiment, a hydrogel composition comprises uncrosslinked chondroitin sulfate polymers, uncrosslinked dermatan sulfate polymers, uncrosslinked keratan sulfate polymers, uncrosslinked heparan polymers, uncrosslinked heparan sulfate polymers, or uncrosslinked hyaluronan polymers.

Aspects of the present specification provide, in part, a hydrogel composition comprising substantially uncrosslinked glycosaminoglycan polymers. As used herein, the term “substantially uncrosslinked” refers to the presence of uncrosslinked glycosaminoglycan polymers in a hydrogel composition disclosed herein at a level of at least 90% by weight of the composition, with the remaining at most 10% by weight of the composition being comprised of other components including crosslinked glycosaminoglycan polymers.

In an embodiment, a hydrogel composition comprises substantially uncrosslinked glycosaminoglycan polymers. In aspects of this embodiment, a hydrogel composition comprises substantially uncrosslinked chondroitin sulfate polymers, substantially uncrosslinked dermatan sulfate polymers, substantially uncrosslinked keratan sulfate polymers, substantially uncrosslinked heparan polymers, substantially uncrosslinked heparan sulfate polymers, or substantially uncrosslinked hyaluronan polymers. In other aspects of this embodiment, a hydrogel composition comprises uncrosslinked glycosaminoglycan polymers where the uncrosslinked glycosaminoglycan polymers represents, e.g., about 20% or less by weight, about 18% or less by weight, about 15% or less by weight, about 12% or less by weight, about 10% or less by weight, about 9% or less by weight, about 8% or less by weight, about 7% or less by weight, about 6% or less by weight, about 5% or less by weight, about 4% or less by weight, about 3% or less by weight, about 2% or less by weight, of the total amount of glycosaminoglycan polymers present in the composition. In yet other aspects of this embodiment, a hydrogel composition comprises uncrosslinked glycosaminoglycan polymers where the uncrosslinked glycosaminoglycan polymers represents, e.g., about 10% to about 20% by weight, about 10% to about 15% by weight, about 5% to about 20% by weight, about 5% to about 15% by weight, about 5% to about 10% by weight, about 2% to about 20% by weight, about 2% to about 15% by weight, about 2% to about 10% by weight, or about 2% to about 5% by weight, of the total amount of glycosaminoglycan polymers present in the composition.

In other aspects of this embodiment, a hydrogel composition comprises uncrosslinked glycosaminoglycan polymers where the uncrosslinked glycosaminoglycan polymers represents, e.g., about 90% or more by weight, about 91% or more by weight, about 92% or more by weight, about 93% or more by weight, about 94% or more by weight, about 95% or more by weight, about 96% or more by weight, about 97% or more by weight, about 98% or more by weight, or about 99% or more, or about 100% by weight, of the total amount of glycosaminoglycan polymers present in the composition. In yet other aspects of this embodiment, a hydrogel composition comprises uncrosslinked glycosaminoglycan polymers where the uncrosslinked glycosaminoglycan polymers represents, e.g., about 90% to about 100% by weight, about 93% to about 100% by weight, about 95% to about 100% by weight, or about 97% to about 100% by weight, of the total amount of glycosaminoglycan polymers present in the composition.

Aspects of the present specification provide, in part, a hydrogel composition that is essentially free of crosslinked glycosaminoglycan polymers. As used herein, the term “essentially free” (or “consisting essentially of”) refers to a hydrogel composition where only trace amounts of cross-linked glycosaminoglycan polymers can be detected.

In an embodiment, a hydrogel composition comprises glycosaminoglycan polymers that are essentially free of crosslinked glycosaminoglycan polymers. In an aspect of this embodiment, a hydrogel composition comprises chondroitin sulfate polymers that are essentially free of crosslinked chondroitin sulfate polymers, dermatan sulfate polymers that are essentially free of crosslinked dermatan sulfate polymers, keratan sulfate polymers that are essentially free of a crosslinked keratan sulfate polymers, heparan polymers that are essentially free of a crosslinked heparan polymers, heparan sulfate polymers that are essentially free of crosslinked heparan sulfate polymers, or hyaluronan polymers that are essentially free of a crosslinked hyaluronan polymers.

Aspects of the present specification provide, in part, a hydrogel composition that is entirely free of a crosslinked glycosaminoglycan polymers. As used herein, the term “entirely free” refers to a hydrogel composition that within the detection range of the instrument or process being used, crosslinked glycosaminoglycan polymers cannot be detected or their presence cannot be confirmed.

In an embodiment, a hydrogel composition comprises glycosaminoglycan polymers that are entirely free of crosslinked glycosaminoglycan polymers. In an aspect of this embodiment, a hydrogel composition comprises chondroitin sulfate polymers that are entirely free of crosslinked chondroitin sulfate polymers, dermatan sulfate polymers that are entirely free of crosslinked dermatan sulfate polymers, keratan sulfate polymers that are entirely free of crosslinked keratan sulfate polymers, heparan polymers that are entirely free of crosslinked heparan polymers, heparan sulfate polymers that are entirely free of crosslinked heparan sulfate polymers, or hyaluronan polymers that are entirely free of crosslinked hyaluronan polymers.

Aspects of the present specification provide, in part, a hydrogel composition comprising a ratio of crosslinked glycosaminoglycan polymers and uncrosslinked glycosaminoglycan polymers. This ratio of crosslinked and uncrosslinked glycosaminoglycan polymers is also known as the gel:fluid ratio. Any gel:fluid ratio is useful in making the hydrogel compositions disclosed herein with the proviso that such ratio produces a hydrogel composition disclosed herein that improves a soft tissue condition as disclosed herein. Non-limiting examples of gel:fluid ratios include 100:0, 98:2, 90:10, 75:25, 70:30, 60:40, 50:50, 40:60, 30:70, 25:75, 10:90; 2:98, and 0:100.

In an embodiment, a hydrogel composition comprises crosslinked glycosaminoglycan polymers and uncrosslinked glycosaminoglycan polymers. In another aspect of this embodiment, a hydrogel composition comprises crosslinked glycosaminoglycan polymers and uncrosslinked glycosaminoglycan polymers where the gel:fluid ratio is sufficient to form a fluid. In other aspects of this embodiment, a hydrogel composition comprises crosslinked glycosaminoglycan polymers and uncrosslinked glycosaminoglycan polymers where the gel:fluid ratio is, e.g., about 0:100, about 1:99, about 2:98, about 3:97, about 4:96, about 5:95, about 6:94, about 7:93, about 8:92, about 9:91, or about 10:90. In yet other aspects of this embodiment, a hydrogel composition comprises crosslinked glycosaminoglycan polymers and uncrosslinked glycosaminoglycan polymers where the gel:fluid ratio is, e.g., at most 1:99, at most 2:98, at most 3:97, at most 4:96, at most 5:95, at most 6:94, at most 7:93, at most 8:92, at most 9:91, or at most 10:90. In still other aspects of this embodiment, a hydrogel composition comprises crosslinked glycosaminoglycan polymers and uncrosslinked glycosaminoglycan polymers where the gel:fluid ratio is, e.g., about 0:100 to about 3:97, about 0:100 to about 5:95, or about 0:100 to about 10:90.

In other aspects of this embodiment, a hydrogel composition comprises crosslinked glycosaminoglycan polymers and uncrosslinked glycosaminoglycan polymers where the gel:fluid ratio is, e.g., about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, about 95:5, about 98:2, or about 100:0. In yet other aspects of this embodiment, a hydrogel composition comprises crosslinked glycosaminoglycan polymers and uncrosslinked glycosaminoglycan polymers where the gel:fluid ratio is, e.g., at most 15:85, at most 20:80, at most 25:75, at most 30:70, at most 35:65, at most 40:60, at most 45:55, at most 50:50, at most 55:45, at most 60:40, at most 65:35, at most 70:30, at most 75:25, at most 80:20, at most 85:15, at most 90:10, at most 95:5, at most 98:2, or at most 100:0. In still other aspects of this embodiment, a hydrogel composition comprises crosslinked glycosaminoglycan polymers and uncrosslinked glycosaminoglycan polymers where the gel:fluid ratio is, e.g., about 10:90 to about 70:30, about 15:85 to about 70:30, about 10:90 to about 55:45, about 80:20 to about 95:5, about 90:10 to about 100:0, about 75:25 to about 100:0, or about 60:40 to about 100:0.

In still another embodiment, a hydrogel composition comprises uncrosslinked glycosaminoglycan polymers where the uncrosslinked glycosaminoglycan polymers are present in an amount sufficient to improve a soft tissue condition as disclosed herein. In aspects of this embodiment, a hydrogel composition comprises uncrosslinked glycosaminoglycan polymers where the uncrosslinked glycosaminoglycan polymers are present at a concentration of, e.g., about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 13.5 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, or about 30 mg/mL. In other aspects of this embodiment, a hydrogel composition comprises uncrosslinked glycosaminoglycan polymers where the uncrosslinked glycosaminoglycan polymers are present at a concentration of, e.g., at least 1 mg/mL, at least 5 mg/mL, at least 10 mg/mL, at least 15 mg/mL, at least 20 mg/mL, at least 25 mg/mL, or at least 30 mg/mL. In yet other aspects of this embodiment, a hydrogel composition comprises uncrosslinked glycosaminoglycan polymers where the uncrosslinked glycosaminoglycan polymers are present at a concentration of, e.g., at most 1 mg/mL, at most 5 mg/mL, at most 10 mg/mL, at most 15 mg/mL, at most 20 mg/mL, at most 25 mg/mL, or at most 30 mg/mL. In still other aspects of this embodiment, a hydrogel composition comprises uncrosslinked glycosaminoglycan polymers where the uncrosslinked glycosaminoglycan polymers are present at a concentration of, e.g., about 7.5 mg/mL to about 19.5 mg/mL, about 8.5 mg/mL to about 18.5 mg/mL, about 9.5 mg/mL to about 17.5 mg/mL, about 10.5 mg/mL to about 16.5 mg/mL, about 11.5 mg/mL to about 15.5 mg/mL, about 12.5 mg/mL to about 14.5 mg/mL, about 20 mg/mL to about 30 mg/mL, about 21 mg/mL to about 29 mg/mL, about 22 mg/mL to about 28 mg/mL, about 23 mg/mL to about 27 mg/mL, or about 24 mg/mL to about 26 mg/mL.

In another embodiment, a hydrogel composition comprises uncrosslinked hyaluronan polymers where the uncrosslinked hyaluronan polymers comprises low molecular weight hyaluronan polymers; high molecular weight hyaluronan polymers; or a combination of both low molecular weight hyaluronan polymers and high molecular weight hyaluronan polymers in various ratios. As used herein, the term “low molecular weight hyaluronan polymer” or “low molecular weight hyaluronan” refers to a hyaluronan polymer that has a molecular weight of less than 1,000,000 Da. Non-limiting examples of a low molecular weight hyaluronan polymers include a hyaluronan polymer of about 200,000 Da, about 300,000 Da, about 400,000 Da, about 500,000 Da, about 600,000 Da, about 700,000 Da, about 800,000 Da, or about 900,000 Da. As used herein, the term “high molecular weight hyaluronan polymer” or “high molecular weight hyaluronan” refers to a hyaluronan polymer that has a molecular weight of 1,000,000 Da or greater. Non-limiting examples of a high molecular weight hyaluronan polymer include a hyaluronan polymer of about 1,500,000 Da, about 2,000,000 Da, about 2,500,000 Da, about 3,000,000 Da, about 3,500,000 Da, about 4,000,000 Da, about 4,500,000 Da, or about 5,000,000 Da.

In other aspects of this embodiment, a hydrogel composition comprises uncrosslinked hyaluronan polymers where the uncrosslinked hyaluronan polymers have a mean molecular weight of, e.g., about 200,000 Da, about 300,000 Da, about 400,000 Da, about 500,000 Da, about 600,000 Da, about 700,000 Da, about 800,000 Da, or about 900,000 Da. In yet other aspects of this embodiment, a hydrogel composition comprises uncrosslinked hyaluronan polymers where the uncrosslinked hyaluronan polymers have a mean molecular weight of, e.g., less than 200,000 Da, less than 300,000 Da, less than 400,000 Da, less than 500,000 Da, less than 600,000 Da, less than 700,000 Da, less than 800,000 Da, less than 900,000 Da, or at most 1,000,000 Da. In still other aspects of this embodiment, a hydrogel composition comprises uncrosslinked hyaluronan polymers where the uncrosslinked hyaluronan polymers have a mean molecular weight of, e.g., about 200,000 Da to about 400,000 Da, about 300,000 Da to about 500,000 Da, about 400,000 Da to about 600,000 Da, about 500,000 Da to about 700,000 Da, about 600,000 Da to about 800,000 Da, about 700,000 Da to about 900,000 Da, about 200,000 Da to about 500,000 Da, about 200,000 Da to about 600,000 Da, about 200,000 Da to about 700,000 Da, about 200,000 Da to about 800,000 Da, about 200,000 Da to about 900,000 Da, about 300,000 Da to about 900,000 Da, about 400,000 Da to about 900,000 Da, about 500,000 Da to about 900,000 Da, about 200,000 Da to less than 1,000,000 Da, about 300,000 Da to less than 1,000,000 Da, about 400,000 Da to less than 1,000,000 Da, or about 500,000 Da to less than 1,000,000 Da.

In other aspects of this embodiment, a hydrogel composition comprises uncrosslinked hyaluronan polymers where the uncrosslinked hyaluronan polymers have a mean molecular weight of, e.g., about 1,000,000 Da, about 1,500,000 Da, about 2,000,000 Da, about 2,500,000 Da, about 3,000,000 Da, about 3,500,000 Da, about 4,000,000 Da, about 4,500,000 Da, or about 5,000,000 Da. In yet other aspects of this embodiment, a hydrogel composition comprises uncrosslinked hyaluronan polymers where the uncrosslinked hyaluronan polymers have a mean molecular weight of, e.g., at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da. In still other aspects of this embodiment, a hydrogel composition comprises uncrosslinked hyaluronan polymers where the uncrosslinked hyaluronan polymers have a mean molecular weight of, e.g., about 1,000,000 Da to about 5,000,000 Da, about 1,500,000 Da to about 5,000,000 Da, about 2,000,000 Da to about 5,000,000 Da, about 2,500,000 Da to about 5,000,000 Da, about 2,000,000 Da to about 3,000,000 Da, about 2,500,000 Da to about 3,500,000 Da, or about 2,000,000 Da to about 4,000,000 Da. In further aspects, a hydrogel composition comprises uncrosslinked hyaluronan polymers where the uncrosslinked hyaluronan polymers have a mean molecular weight of, e.g., greater than 2,000,000 Da and less than about 3,000,000 Da, greater than 2,000,000 Da and less than about 3,500,000 Da, greater than 2,000,000 Da and less than about 4,000,000 Da, greater than 2,000,000 Da and less than about 4,500,000 Da, greater than 2,000,000 Da and less than about 5,000,000 Da.

In yet other aspects of this embodiment, a hydrogel composition comprises uncrosslinked hyaluronan polymers where the uncrosslinked hyaluronan polymers comprises a combination of both low molecular weight hyaluronan polymers and high molecular weight hyaluronan polymers in a ratio of about 20:1, about 15:1, about 10:1, about 5:1, about 1:1, about 1:5 about 1:10, about 1:15, or about 1:20.

In another embodiment, a hydrogel composition comprises crosslinked hyaluronan polymers where the crosslinked hyaluronan polymers comprises low molecular weight hyaluronan polymers; high molecular weight hyaluronan polymers; or a combination of both low molecular weight hyaluronan polymers and high molecular weight hyaluronan polymers in various ratios.

In other aspects of this embodiment, a hydrogel composition comprises crosslinked hyaluronan polymers where the crosslinked hyaluronan polymers have a mean molecular weight of, e.g., about 200,000 Da, about 300,000 Da, about 400,000 Da, about 500,000 Da, about 600,000 Da, about 700,000 Da, about 800,000 Da, or about 900,000 Da. In yet other aspects of this embodiment, a hydrogel composition comprises crosslinked hyaluronan polymers where the crosslinked hyaluronan polymers have a mean molecular weight of, e.g., less than 200,000 Da, less than 300,000 Da, less than 400,000 Da, less than 500,000 Da, less than 600,000 Da, less than 700,000 Da, less than 800,000 Da, less than 900,000 Da, or at most 1,000,000 Da. In still other aspects of this embodiment, a hydrogel composition comprises crosslinked hyaluronan polymers where the crosslinked hyaluronan polymers have a mean molecular weight of, e.g., about 200,000 Da to about 400,000 Da, about 300,000 Da to about 500,000 Da, about 400,000 Da to about 600,000 Da, about 500,000 Da to about 700,000 Da, about 600,000 Da to about 800,000 Da, about 700,000 Da to about 900,000 Da, about 200,000 Da to about 500,000 Da, about 200,000 Da to about 600,000 Da, about 200,000 Da to about 700,000 Da, about 200,000 Da to about 800,000 Da, about 200,000 Da to about 900,000 Da, about 300,000 Da to about 900,000 Da, about 400,000 Da to about 900,000 Da, about 500,000 Da to about 900,000 Da, about 200,000 Da to less than 1,000,000 Da, about 300,000 Da to less than 1,000,000 Da, about 400,000 Da to less than 1,000,000 Da, or about 500,000 Da to less than 1,000,000 Da.

In other aspects of this embodiment, a hydrogel composition comprises crosslinked hyaluronan polymers where the crosslinked hyaluronan polymers have a mean molecular weight of, e.g., about 1,000,000 Da, about 1,500,000 Da, about 2,000,000 Da, about 2,500,000 Da, about 3,000,000 Da, about 3,500,000 Da, about 4,000,000 Da, about 4,500,000 Da, or about 5,000,000 Da. In yet other aspects of this embodiment, a hydrogel composition comprises crosslinked hyaluronan polymers where the crosslinked hyaluronan polymers have a mean molecular weight of, e.g., at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da. In still other aspects of this embodiment, a hydrogel composition comprises crosslinked hyaluronan polymers where the crosslinked hyaluronan polymers have a mean molecular weight of, e.g., about 1,000,000 Da to about 5,000,000 Da, about 1,500,000 Da to about 5,000,000 Da, about 2,000,000 Da to about 5,000,000 Da, about 2,500,000 Da to about 5,000,000 Da, about 2,000,000 Da to about 3,000,000 Da, about 2,500,000 Da to about 3,500,000 Da, or about 2,000,000 Da to about 4,000,000 Da. In further aspects, a hydrogel composition comprises crosslinked hyaluronan polymers where the crosslinked hyaluronan polymers have a mean molecular weight of, e.g., greater than 2,000,000 Da and less than about 3,000,000 Da, greater than 2,000,000 Da and less than about 3,500,000 Da, greater than 2,000,000 Da and less than about 4,000,000 Da, greater than 2,000,000 Da and less than about 4,500,000 Da, greater than 2,000,000 Da and less than about 5,000,000 Da.

In yet other aspects of this embodiment, a hydrogel composition comprises crosslinked hyaluronan polymers where the crosslinked hyaluronan polymers comprises a combination of both low molecular weight hyaluronan polymers and high molecular weight hyaluronan polymers in a ratio of about 20:1, about 15:1, about 10:1, about 5:1, about 1:1, about 1:5 about 1:10, about 1:15, or about 1:20.

Aspects of the present specification provide, in part, hydrogel composition comprising glycosaminoglycan polymers coated in a PEG-based pendant. As a biocompatible, inert, and hydrophilic polymer, PEG offers good degradation resistance to both crosslinked and non-crosslinked glycosaminoglycan polymers, thereby enhancing the in vivo longevity of the polymers. Additionally, crosslinked glycosaminoglycan polymers disclosed herein may be first processed into particles and then coated with a PEG-based pendant. A PEG-based pendant may be coated onto glycosaminoglycan polymers as disclosed herein to any desired thickness, with the proviso that the thickness provides degradation resistance to the polymers.

In aspects of this embodiment, a hydrogel composition comprises glycosaminoglycan polymers are coated with a PEG-based pendant to a thickness of, e.g., about 2 nm, about 5 nm, about 10 nm, about 15 nm, about 20 nm, about 25 nm, about 30 nm, about 35 nm, about 40 nm, about 45 nm, or about 50 nm. In other aspects of this embodiment, a hydrogel composition comprises glycosaminoglycan polymers are coated with a PEG-based pendant to a thickness of, e.g., at least 2 nm, at least 5 nm, at least 10 nm, at least 15 nm, at least 20 nm, at least 25 nm, at least 30 nm, at least 35 nm, at least 40 nm, at least 45 nm, or at least 50 nm. In yet other aspects of this embodiment, a hydrogel composition comprises glycosaminoglycan polymers are coated with a PEG-based pendant to a thickness of, e.g., about 2 nm to about 5 nm, about 2 nm to about 10 nm, about 2 nm to about 15 nm, about 2 nm to about 20 nm, about 2 nm to about 25 nm, about 2 nm to about 30 nm, about 2 nm to about 35 nm, about 2 nm to about 40 nm, about 2 nm to about 45 nm, about 2 nm to about 50 nm, about 5 nm to about 10 nm, about 5 nm to about 15 nm, about 5 nm to about 20 nm, about 5 nm to about 25 nm, about 5 nm to about 30 nm, about 5 nm to about 35 nm, about 5 nm to about 40 nm, about 5 nm to about 45 nm, about 5 nm to about 50 nm, about 10 nm to about 15 nm, about 10 nm to about 20 nm, about 10 nm to about 25 nm, about 10 nm to about 30 nm, about 10 nm to about 35 nm, about 10 nm to about 40 nm, about 10 nm to about 45 nm, about 10 nm to about 50 nm, about 20 nm to about 25 nm, about 20 nm to about 30 nm, about 20 nm to about 35 nm, about 20 nm to about 40 nm, about 20 nm to about 45 nm, about 20 nm to about 50 nm, about 25 nm to about 30 nm, about 25 nm to about 35 nm, about 25 nm to about 40 nm, about 25 nm to about 45 nm, or about 25 nm to about 50 nm.

In another embodiment, a hydrogel composition comprising PEG-based pendant coated glycosaminoglycan polymers as disclosed herein exhibit resistance to degradation. In aspects of this embodiment, a hydrogel composition comprising PEG-based pendant coated glycosaminoglycan polymers as disclosed herein exhibit resistance to degradation that is, e.g., about 10% more, about 20% more, about 30% more, about 40% more, about 50% more, about 60% more, about 70% more, about 80% more, about 90% more, about 100% more, about 125% more, about 150% more, about 175% more, about 200% more, about 225% more, about 250% more, about 275% more, or about 300% more than the same or similar glycosaminoglycan polymers, but without the PEG-based pendant coating. In other aspects of this embodiment, a hydrogel composition comprising PEG-based pendant coated glycosaminoglycan polymers as disclosed herein exhibit resistance to degradation that is at least 10% more, at least 20% more, at least 30% more, at least 40% more, at least 50% more, at least 60% more, at least 70% more, at least 80% more, at least 90% more, at least 100% more, at least 125% more, at least 150% more, at least 175% more, at least 200% more, at least 225% more, at least 250% more, at least 275% more, or at least 300% more than the same or similar glycosaminoglycan polymers, but without the PEG-based pendant coating. In aspects of this embodiment, a hydrogel composition comprising PEG-based pendant coated glycosaminoglycan polymers as disclosed herein exhibit resistance to degradation that is, e.g., about 10% more to about 100% more, about 10% more to about 200% more, about 10% more to about 300% more, about 50% more to about 100% more, about 100% more to about 150% more, about 150% more to about 200% more, about 200% more to about 250% more, or about 250% more to about 300% more than the same or similar glycosaminoglycan polymers, but without the PEG-based pendant coating.

Aspects of the present specification provide, in part, a hydrogel comprising crosslinked glycosaminoglycan polymers processed into particles. Hydrogels comprising crosslinked glycosaminoglycan polymers may be further processed by pulverizing, sieving, milling or otherwise breaking apart the material into particles having a size from about 10 μm to about 1000 μm in approximate diameter. For example, hydrogel processing may be accomplished by means of a forced sieving of bulk crosslinked polymers through a series of stainless steel cloth sieves of decreasing pore sizes or by pulverizing by mechanical grinding technique. The hydrogel compositions disclosed herein may also be processed into cylindrical or hair-shaped particles as described in Stroumpoulis and Guillen, Hair-Shaped Hydrogels for Soft Tissue Augmentation, U.S. patent application, Ser. No. 12/753,361, filed on Apr. 2, 2010, which is hereby incorporated by reference in its entirety. The resulting hydrogel composition may then be purified, such as, e.g., by dialysis.

In aspects of this embodiment, a hydrogel composition may be sized by passing the material through a mesh screen only once or for a plurality of times. In aspects of this embodiment, a hydrogel composition may be sized by passing the material through a 25 μm, a 43 μm, a 60 μm, or a 105 μm, mesh screen only once. In other aspects of this embodiment, a hydrogel composition may be sized by passing the material through a 25 μm, a 43 μm, a 60 μm, or a 105 μm, mesh screen twice, three times, four times, five times, six times, seven times, eight times, nine times, or ten times. In other aspects of this embodiment, a hydrogel composition may be sized by passing the material through a 25 μm, a 43 μm, a 60 μm, or a 105 μm, mesh screen at least twice, at least three times, at least four times, at least five times, at least six times, at least seven times, at least eight times, at least nine times, or at least ten times. In other aspects of this embodiment, a hydrogel composition may be sized by passing the material through a 25 μm, a 43 μm, a 60 μm, or a 105 μm, mesh screen at least twice to five times, at least three times to six times, at least four to seven times, at least five to five eight, or at least six to nine times.

In aspects of this embodiment, a hydrogel composition comprising crosslinked glycosaminoglycan polymers as disclosed herein has crosslinked glycosaminoglycan polymers with a mean particle size of, e.g., about 50 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, or about 800 μm. In yet other aspects of this embodiment, a hydrogel composition comprising crosslinked glycosaminoglycan polymers as disclosed herein has crosslinked glycosaminoglycan polymers with a mean particle size of, e.g., at most 50 μm, at most 100 μm, at most 150 μm, at most 200 μm, at most 250 μm, at most 300 μm, at most 350 μm, at most 400 μm, at most 450 μm, at most 500 μm, at most 550 μm, at most 600 μm, at most 650 μm, at most 700 μm, at most 750 μm, or at most 800 μm. In still other aspects of this embodiment, a hydrogel composition comprising crosslinked glycosaminoglycan polymers as disclosed herein has crosslinked glycosaminoglycan polymers with a mean particle size of, e.g., about 50 μm to about 150 μm, about 100 μm to about 200 μm, about 200 μm to about 300 μm, about 300 μm to about 400 μm, about 400 μm to about 500 μm, about 500 μm to about 600 μm, about 600 μm to about 700 μm, about 700 μm to about 800 μm, about 50 μm to about 250 μm, about 100 μm to about 300 μm, about 100 μm to about 400 μm, about 200 μm to about 400 μm, about 200 μm to about 500 μm, about 200 μm to about 600 μm, about 200 μm to about 700 μm, about 200 μm to about 800 μm, about 300 μm to about 500 μm, about 300 μm to about 600 μm, about 300 μm to about 700 μm, or about 300 μm to about 800 μm.

The processed hydrogel compositions comprising crosslinked glycosaminoglycan polymers may, or may not, be mixed with anther gel phase or a carrier phase to form an injectable or topical substance like a solution, oil, lotion, gel, ointment, cream, slurry, salve, or paste. As such, the disclosed hydrogel compositions can be monophasic or multiphasic compositions. The volume of another gel phase or carrier phase added to the particles will depend upon the desired physical properties of the resultant composition including dose delivery, viscosity, injectability, and desired in vivo behavioral characteristics.

As used herein, the term “carrier phase” is synonymous with “carrier” and refers to a material used to increase fluidity of a hydrogel composition disclosed herein. A carrier is advantageously a physiologically-acceptable carrier and may include one or more conventional excipients useful in pharmaceutical compositions. As used herein, the term “a physiologically-acceptable carrier” refers to a carrier in accord with, or characteristic of, the normal functioning of a living organism. As such, administration of a hydrogel composition comprising crosslinked glycosaminoglycan polymers and a carrier has substantially no long term or permanent detrimental effect when administered to a mammal. The present compositions include a carrier where a major of the volume is water, saline, or a buffered-saline. However, other useful carriers include any physiologically tolerable buffer, serum or other protein solutions. The volume of carrier per volume of crosslinked glycosaminoglycan polymer particles may be increased or decreased in a range between 0% to 100% depending upon the desired physical properties of the resultant composition including dose delivery, viscosity, injectability, and desired in vivo behavioral characteristics. This carrier is then mixed with the particles until achieving a “uniform” consistency which may be termed an emulsion or suspension. Advantages derived from adding a carrier to a hydrogel composition disclosed herein include decreased viscosity in the extracellular in vivo microenvironment; release of local mechanical stress loading after drug delivery platform administration; and improved ionic composition resulting in improved biocompatibility.

Aspects of the present specification provide, in part, a hydrogel composition that may, or may not, comprise an anesthetic agent. An anesthetic agent is preferably a local anesthetic agent, i.e., an anesthetic agent that causes a reversible local anesthesia and a loss of nociception, such as, e.g., aminoamide local anesthetics and aminoester local anesthetics. The amount of an anesthetic agent included in a hydrogel composition disclosed herein is an amount effective to mitigate pain experienced by an individual upon administration of the composition. As such, the amount of an anesthetic agent included in a hydrogel composition disclosed herein is between about 0.1% (w/w) to about 5% (w/w) by weight of the total composition. Non-limiting examples of anesthetic agents include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, combinations thereof, and salts thereof. Non-limiting examples of aminoester local anesthetics include procaine, chloroprocaine, cocaine, cyclomethycaine, cimethocaine (larocaine), propoxycaine, procaine (novocaine), proparacaine, tetracaine (amethocaine). Non-limiting examples of aminoamide local anesthetics include articaine, bupivacaine, cinchocaine (dibucaine), etidocaine, levobupivacaine, lidocaine (lignocaine), mepivacaine, piperocaine, prilocaine, ropivacaine, and trimecaine. A non-limiting example of a combination local anesthetic is lidocaine/prilocaine (EMLA).

Aspects of the present specification provide, in part, a hydrogel composition comprising crosslinked glycosaminoglycan polymers as disclosed herein that exhibits a complex modulus, an elastic modulus, a viscous modulus and/or a tan δ. The compositions as disclosed herein are viscoelastic in that the composition has an elastic component (solid-like such as, e.g., crosslinked glycosaminoglycan polymers) and a viscous component (liquid-like such as, e.g., uncrosslinked glycosaminoglycan polymers or a carrier phase) when a force is applied (stress, deformation). The rheological attribute that described this property is the complex modulus (G*), which defines a composition's total resistance to deformation. The complex modulus is a complex number with a real and imaginary part: G*=G′+iG″. The absolute value of G* is Abs(G*)=Sqrt(G′²+G″²). The complex modulus can be defined as the sum of the elastic modulus (G′) and the viscous modulus (G”). Falcone, et al., Temporary Polysaccharide Dermal Fillers: A Model for Persistence Based on Physical Properties, Dermatol Surg. 35(8): 1238-1243 (2009); Tezel, supra, 2008; Kablik, supra, 2009; Beasley, supra, 2009; each of which is hereby incorporated by reference in its entirety. Elastic modulus characterizes the firmness of a composition and is also known as the storage modulus because it describes the storage of energy from the motion of the composition. The elastic modulus describes the interaction between elasticity and strength (G′=stress/strain) and, as such, provides a quantitative measurement of a composition's hardness or softness. Although depending on the speed at which the force is applied, a stiffer composition will have a higher elastic modulus and it will take a greater force to deform the material a given distance, such as, e.g., an injection.

Viscous modulus is also known as the loss modulus because it describes the energy that is lost as viscous dissipation. Tan δ is the ratio of the viscous modulus and the elastic modulus, tan δ=G″/G′. Falcone, supra, 2009. For tan δ values disclosed in the present specification, a tan δ is obtained from the dynamic modulus at a frequency of 0.628 rad/s. A lower tan δ corresponds to a stiffer, harder, or more elastic composition.

Thus, in an embodiment, a hydrogel composition comprising crosslinked glycosaminoglycan polymers as disclosed herein exhibits a complex modulus. In aspects of this embodiment, a hydrogel composition exhibits a complex modulus of, e.g., about 25 Pa, about 50 Pa, about 75 Pa, about 100 Pa, about 125 Pa, about 150 Pa, about 175 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, or about 800 Pa. In other aspects of this embodiment, a hydrogel composition exhibits a complex modulus of, e.g., at most 25 Pa, at most 50 Pa, at most 75 Pa, at most 100 Pa, at most 125 Pa, at most 150 Pa, at most 175 Pa, at most 200 Pa, at most 250 Pa, at most 300 Pa, at most 350 Pa, at most 400 Pa, at most 450 Pa, at most 500 Pa, at most 550 Pa, at most 600 Pa, at most 650 Pa, at most 700 Pa, at most 750 Pa, or at most 800 Pa. In yet other aspects of this embodiment, a hydrogel composition exhibits a complex modulus of, e.g., about 25 Pa to about 150 Pa, about 25 Pa to about 300 Pa, about 25 Pa to about 500 Pa, about 25 Pa to about 800 Pa, about 125 Pa to about 300 Pa, about 125 Pa to about 500 Pa, or about 125 Pa to about 800 Pa.

In another embodiment, a hydrogel composition comprising crosslinked glycosaminoglycan polymers as disclosed herein exhibits an elastic modulus. In aspects of this embodiment, a hydrogel composition exhibits an elastic modulus of, e.g., about 25 Pa, about 50 Pa, about 75 Pa, about 100 Pa, about 125 Pa, about 150 Pa, about 175 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, about 800 Pa, about 850 Pa, about 900 Pa, about 950 Pa, about 1,000 Pa, about 1,200 Pa, about 1,300 Pa, about 1,400 Pa, about 1,500 Pa, about 1,600 Pa, about 1700 Pa, about 1800 Pa, about 1900 Pa, about 2,000 Pa, about 2,100 Pa, about 2,200 Pa, about 2,300 Pa, about 2,400 Pa, or about 2,500 Pa. In other aspects of this embodiment, a hydrogel composition exhibits an elastic modulus of, e.g., at least 25 Pa, at least 50 Pa, at least 75 Pa, at least 100 Pa, at least 125 Pa, at least 150 Pa, at least 175 Pa, at least 200 Pa, at least 250 Pa, at least 300 Pa, at least 350 Pa, at least 400 Pa, at least 450 Pa, at least 500 Pa, at least 550 Pa, at least 600 Pa, at least 650 Pa, at least 700 Pa, at least 750 Pa, at least 800 Pa, at least 850 Pa, at least 900 Pa, at least 950 Pa, at least 1,000 Pa, at least 1,200 Pa, at least 1,300 Pa, at least 1,400 Pa, at least 1,500 Pa, at least 1,600 Pa, at least 1700 Pa, at least 1800 Pa, at least 1900 Pa, at least 2,000 Pa, at least 2,100 Pa, at least 2,200 Pa, at least 2,300 Pa, at least 2,400 Pa, or at least 2,500 Pa. In yet other aspects of this embodiment, a hydrogel composition exhibits an elastic modulus of, e.g., at most 25 Pa, at most 50 Pa, at most 75 Pa, at most 100 Pa, at most 125 Pa, at most 150 Pa, at most 175 Pa, at most 200 Pa, at most 250 Pa, at most 300 Pa, at most 350 Pa, at most 400 Pa, at most 450 Pa, at most 500 Pa, at most 550 Pa, at most 600 Pa, at most 650 Pa, at most 700 Pa, at most 750 Pa, at most 800 Pa, at most 850 Pa, at most 900 Pa, at most 950 Pa, at most 1,000 Pa, at most 1,200 Pa, at most 1,300 Pa, at most 1,400 Pa, at most 1,500 Pa, or at most 1,600 Pa. In still other aspects of this embodiment, a hydrogel composition exhibits an elastic modulus of, e.g., about 25 Pa to about 150 Pa, about 25 Pa to about 300 Pa, about 25 Pa to about 500 Pa, about 25 Pa to about 800 Pa, about 125 Pa to about 300 Pa, about 125 Pa to about 500 Pa, about 125 Pa to about 800 Pa, about 500 Pa to about 1,600 Pa, about 600 Pa to about 1,600 Pa, about 700 Pa to about 1,600 Pa, about 800 Pa to about 1,600 Pa, about 900 Pa to about 1,600 Pa, about 1,000 Pa to about 1,600 Pa, about 1,100 Pa to about 1,600 Pa, about 1,200 Pa to about 1,600 Pa, about 500 Pa to about 2,500 Pa, about 1,000 Pa to about 2,500 Pa, about 1,500 Pa to about 2,500 Pa, about 2,000 Pa to about 2,500 Pa, about 1,300 Pa to about 1,600 Pa, about 1,400 Pa to about 1,700 Pa, about 1,500 Pa to about 1,800 Pa, about 1,600 Pa to about 1,900 Pa, about 1,700 Pa to about 2,000 Pa, about 1,800 Pa to about 2,100 Pa, about 1,900 Pa to about 2,200 Pa, about 2,000 Pa to about 2,300 Pa, about 2,100 Pa to about 2,400 Pa, or about 2,200 Pa to about 2,500 Pa.

In another embodiment, a hydrogel composition comprising crosslinked glycosaminoglycan polymers as disclosed herein exhibits a viscous modulus. In aspects of this embodiment, a hydrogel composition exhibits a viscous modulus of, e.g., about 10 Pa, about 20 Pa, about 30 Pa, about 40 Pa, about 50 Pa, about 60 Pa, about 70 Pa, about 80 Pa, about 90 Pa, about 100 Pa, about 150 Pa, about 200 Pa, about 250 Pa, about 300 Pa, about 350 Pa, about 400 Pa, about 450 Pa, about 500 Pa, about 550 Pa, about 600 Pa, about 650 Pa, or about 700 Pa. In other aspects of this embodiment, a hydrogel composition exhibits a viscous modulus of, e.g., at most 10 Pa, at most 20 Pa, at most 30 Pa, at most 40 Pa, at most 50 Pa, at most 60 Pa, at most 70 Pa, at most 80 Pa, at most 90 Pa, at most 100 Pa, at most 150 Pa, at most 200 Pa, at most 250 Pa, at most 300 Pa, at most 350 Pa, at most 400 Pa, at most 450 Pa, at most 500 Pa, at most 550 Pa, at most 600 Pa, at most 650 Pa, or at most 700 Pa. In yet other aspects of this embodiment, a hydrogel composition exhibits a viscous modulus of, e.g., about 10 Pa to about 30 Pa, about 10 Pa to about 50 Pa, about 10 Pa to about 100 Pa, about 10 Pa to about 150 Pa, about 70 Pa to about 100 Pa, about 50 Pa to about 350 Pa, about 150 Pa to about 450 Pa, about 250 Pa to about 550 Pa, about 350 Pa to about 700 Pa, about 50 Pa to about 150 Pa, about 100 Pa to about 200 Pa, about 150 Pa to about 250 Pa, about 200 Pa to about 300 Pa, about 250 Pa to about 350 Pa, about 300 Pa to about 400 Pa, about 350 Pa to about 450 Pa, about 400 Pa to about 500 Pa, about 450 Pa to about 550 Pa, about 500 Pa to about 600 Pa, about 550 Pa to about 650 Pa, or about 600 Pa to about 700 Pa.

In another embodiment, a hydrogel composition comprising crosslinked glycosaminoglycan polymers as disclosed herein exhibits a hardness. In aspects of this embodiment, a hydrogel composition exhibits a hardness of, e.g., about 25 Pa, about 50 Pa, about 75 Pa, about 100 Pa, about 125 Pa, about 150 Pa, about 175 Pa, about 200 Pa, about 250 Pa, about 300 Pa, about 350 Pa, about 400 Pa, about 450 Pa, about 500 Pa, about 550 Pa, about 600 Pa, about 650 Pa, about 700 Pa, about 750 Pa, or about 800 Pa. In other aspects of this embodiment, a hydrogel composition exhibits a hardness of, e.g., at least 25 Pa, at least 50 Pa, at least 75 Pa, at least 100 Pa, at least 125 Pa, at least 150 Pa, at least 175 Pa, at least 200 Pa, at least 250 Pa, at least 300 Pa, at least 350 Pa, at least 400 Pa, at least 450 Pa, at least 500 Pa, at least 550 Pa, at least 600 Pa, at least 650 Pa, at least 700 Pa, at least 750 Pa, or at least 800 Pa. In yet other aspects of this embodiment, a hydrogel composition exhibits a hardness of, e.g., about 100 Pa to about 150 Pa, about 100 Pa to about 300 Pa, about 100 Pa to about 500 Pa, about 100 Pa to about 800 Pa, about 125 Pa to about 300 Pa, about 125 Pa to about 500 Pa, or about 125 Pa to about 800 Pa.

In another embodiment, a hydrogel composition comprising crosslinked glycosaminoglycan polymers as disclosed herein exhibits a tan δ. In aspects of this embodiment, a hydrogel composition exhibits a tan δ of, e.g., about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, or about 2.5. In other aspects of this embodiment, a hydrogel composition exhibits a tan δ of, e.g., at most 0.1, at most 0.2, at most 0.3, at most 0.4, at most 0.5, at most 0.6, at most 0.7, at most 0.8, at most 0.9, at most 1.0, at most 1.1, at most 1.2, at most 1.3, at most 1.4, at most 1.5, at most 1.6, at most 1.7, at most 1.8, at most 1.9, at most 2.0, at most 2.1, at most 2.2, at most 2.3, at most 2.4, or at most 2.5. In yet other aspects of this embodiment, a hydrogel composition exhibits a tan δ of, e.g., about 0.1 to about 0.3, about 0.3 to about 0.5, about 0.5 to about 0.8, about 1.1 to about 1.4, about 1.4 to about 1.7, about 0.3 to about 0.6, about 0.1 to about 0.5, about 0.5 to about 0.9, about 0.1 to about 0.6, about 0.1 to about 1.0, about 0.5 to about 1.5, about 1.0 to about 2.0, or about 1.5 to about 2.5.

Aspects of the present specification provide, in part, a hydrogel composition comprising crosslinked glycosaminoglycan polymers as disclosed herein that exhibits a dynamic viscosity. Viscosity is resistance of a fluid to shear or flow caused by either shear stress or tensile stress. Viscosity describes a fluid's internal resistance to flow caused by intermolecular friction exerted when layers of fluids attempt to slide by one another and may be thought of as a measure of fluid friction. The less viscous the fluid, the greater its ease of movement (fluidity).

Viscosity can be defined in two ways; dynamic viscosity (μ, although η is sometimes used) or kinematic viscosity (v). Dynamic viscosity, also known as absolute or complex viscosity, is the tangential force per unit area required to move one horizontal plane with respect to the other at unit velocity when maintained a unit distance apart by the fluid. The SI physical unit of dynamic viscosity is the Pascal-second (Pa·s), which is identical to N·m−2·s. Dynamic viscosity can be expressed as τ=μ dvx/dz, where τ=shearing stress, μ=dynamic viscosity, and dvx/dz is the velocity gradient over time. For example, if a fluid with a viscosity of one Pa·s is placed between two plates, and one plate is pushed sideways with a shear stress of one Pascal, it moves a distance equal to the thickness of the layer between the plates in one second. Dynamic viscosity symbolize by is also used, is measured with various types of rheometers, devices used to measure the way in which a liquid, suspension or slurry flows in response to applied forces.

Kinematic viscosity (v) is the ratio of dynamic viscosity to density, a quantity in which no force is involved and is defined as follows: v=μ/ρ, where μ is the dynamic viscosity ρ is density with the SI unit of kg/m³. Kinematic viscosity is usually measured by a glass capillary viscometer as has an SI unit of m²/s.

The viscosity of a material is highly temperature dependent and for either dynamic or kinematic viscosity to be meaningful, the reference temperature must be quoted. For the viscosity values disclosed herein, a dynamic viscosity is measured at 1 Pa with a cone/plane geometry 2°/40 cm and a temperature of 20° C. Examples of the dynamic viscosity of various fluids at 20° C. is as follows: water is about 1.0×10⁻³ Pa·s, blood is about 3-4×10⁻³ Pa·s, vegetable oil is about 60-85×10⁻³ Pa·s, motor oil SE 30 is about 0.2 Pa·s, glycerin is about 1.4 Pa·s, maple syrup is about 2-3 Pa·s, honey is about 10 Pa·s, chocolate syrup is about 10-25 Pa·s, peanut butter is about 150-250 Pa·s, lard is about 1,000 Pa·s, vegetable shortening is about 1,200 Pa·s, and tar is about 30,000 Pa·s.

In aspects of this embodiment, a hydrogel composition comprising crosslinked glycosaminoglycan polymers as disclosed herein exhibits a dynamic viscosity of, e.g., about 10 Pa·s, about 20 Pa·s, about 30 Pa·s, about 40 Pa·s, about 50 Pa·s, about 60 Pa·s, about 70 Pa·s, about 80 Pa·s, about 90 Pa·s, about 100 Pa·s, about 125 Pa·s, about 150 Pa·s, about 175 Pa·s, about 200 Pa·s, about 225 Pa·s, about 250 Pa·s, about 275 Pa·s, about 300 Pa·s, about 400 Pa·s, about 500 Pa·s, about 600 Pa·s, about 700 Pa·s, about 750 Pa·s, about 800 Pa·s, about 900 Pa·s, about 1,000 Pa·s, about 1,100 Pa·s, or about 1,200 Pa·s. In other aspects of this embodiment, a hydrogel composition comprising crosslinked glycosaminoglycan polymers as disclosed herein exhibits a dynamic viscosity of, e.g., at most 10 Pa·s, at most 20 Pa·s, at most 30 Pa·s, at most 40 Pa·s, at most 50 Pa·s, at most 60 Pa·s, at most 70 Pa·s, at most 80 Pa·s, at most 90 Pa·s, at most 100 Pa·s, at most 125 Pa·s, at most 150 Pa·s, at most 175 Pa·s, at most 200 Pa·s, at most 225 Pa·s, at most 250 Pa·s, at most 275 Pa·s, at most 300 Pa·s, at most 400 Pa·s, at most 500 Pa·s, at most 600 Pa·s, at most 700 Pa·s, at most 750 Pa·s, at most 800 Pa·s, at most 900 Pa·s, or at most 1000 Pa·s. In yet other aspects of this embodiment, a hydrogel composition comprising crosslinked glycosaminoglycan polymers as disclosed herein exhibits a dynamic viscosity of, e.g., about 10 Pa·s to about 100 Pa·s, about 10 Pa·s to about 150 Pa·s, about 10 Pa·s to about 250 Pa·s, about 50 Pa·s to about 100 Pa·s, about 50 Pa·s to about 150 Pa·s, about 50 Pa·s to about 250 Pa·s, about 100 Pa·s to about 500 Pa·s, about 100 Pa·s to about 750 Pa·s, about 100 Pa·s to about 1,000 Pa·s, about 100 Pa·s to about 1,200 Pa·s, about 300 Pa·s to about 500 Pa·s, about 300 Pa·s to about 750 Pa·s, about 300 Pa·s to about 1,000 Pa·s, or about 300 Pa·s to about 1,200 Pa·s.

Aspects of the present specification provide, in part, a hydrogel composition comprising crosslinked glycosaminoglycan polymers as disclosed herein that is injectable. As used herein, the term “injectable” refers to a material having the properties necessary to administer the composition into a soft tissue part, area and/or region of an individual using an injection device with a fine needle. As used herein, the term “fine needle” refers to a needle that is 22 gauge or smaller.

In aspect of this embodiment, a hydrogel composition comprising crosslinked glycosaminoglycan polymers as disclosed herein is injectable through a fine needle. In other aspects of this embodiment, a hydrogel composition comprising crosslinked glycosaminoglycan polymers as disclosed herein is injectable through a needle of, e.g., about 22 gauge, about 27 gauge, about 30 gauge, or about 32 gauge. In yet other aspects of this embodiment, a hydrogel composition comprising crosslinked glycosaminoglycan polymers as disclosed herein is injectable through a needle of, e.g., 22 gauge or smaller, 27 gauge or smaller, 30 gauge or smaller, or 32 gauge or smaller. In still other aspects of this embodiment, a hydrogel composition comprising crosslinked glycosaminoglycan polymers as disclosed herein is injectable through a needle of, e.g., about 22 gauge to about 32 gauge, about 22 gauge to about 27 gauge, or about 27 gauge to about 32 gauge.

In aspects of this embodiment, a hydrogel composition comprising crosslinked glycosaminoglycan polymers as disclosed herein can be injected through a 27 gauge needle with an extrusion force of about 60 N, about 55 N, about 50 N, about 45 N, about 40 N, about 35 N, about 30 N, about 25 N, about 20 N, about 15 N, or about 10 N. In other aspects of this embodiment, a hydrogel composition comprising crosslinked glycosaminoglycan polymers as disclosed herein can be injected through a 27 gauge needle with an extrusion force of about 60 N or less, about 55 N or less, about 50 N or less, about 45 N or less, about 40 N or less, about 35 N or less, about 30 N or less, about 25 N or less, about 20 N or less, about 15 N or less, about 10 N or less, or about 5 N or less.

In aspects of this embodiment, a hydrogel composition comprising crosslinked glycosaminoglycan polymers as disclosed herein can be injected through a 32 gauge needle with an extrusion force of about 60 N, about 55 N, about 50 N, about 45 N, about 40 N, about 35 N, about 30 N, about 25 N, about 20 N, about 15 N, or about 10 N. In other aspects of this embodiment, a hydrogel composition comprising crosslinked glycosaminoglycan polymers as disclosed herein can be injected through a 32 gauge needle with an extrusion force of about 60 N or less, about 55 N or less, about 50 N or less, about 45 N or less, about 40 N or less, about 35 N or less, about 30 N or less, about 25 N or less, about 20 N or less, about 15 N or less, about 10 N or less, or about 5 N or less.

Aspects of the present specification provide, in part, a hydrogel composition comprising crosslinked glycosaminoglycan polymers as disclosed herein that exhibits cohesivity. Cohesivity, also referred to as cohesion cohesive attraction, cohesive force, or compression force is a physical property of a material, caused by the intermolecular attraction between like-molecules within the material that acts to unite the molecules. Covesivity is expressed in terms of grams-force (gmf). A composition should be sufficiently cohesive as to remain localized to a site of administration. Additionally, in certain applications, a sufficient cohesiveness is important for a composition to retain its shape, and thus functionality, in the event of mechanical load cycling. As such, in one embodiment, a hydrogel composition comprising crosslinked glycosaminoglycan polymers as disclosed herein exhibits cohesivity, on par with water. In yet another embodiment, a hydrogel composition comprising crosslinked glycosaminoglycan polymers as disclosed herein exhibits sufficient cohesivity to remain localized to a site of administration. In still another embodiment, a hydrogel composition comprising crosslinked glycosaminoglycan polymers as disclosed herein exhibits sufficient cohesivity to retain its shape. In a further embodiment, a hydrogel composition comprising crosslinked glycosaminoglycan polymers as disclosed herein exhibits sufficient cohesivity to retain its shape and functionality.

In aspects of this embodiment, a hydrogel composition comprising crosslinked glycosaminoglycan polymers as disclosed herein has a cohesivity of, e.g., about 10 gmf, about 20 gmf, about 30 gmf, about 40 gmf, about 50 gmf, about 60 gmf, about 70 gmf, about 80 gmf, about 90 gmf, about 100 gmf, about 150 gmf, or about 200 gmf. In other aspects of this embodiment, a hydrogel composition comprising crosslinked glycosaminoglycan polymers as disclosed herein has a cohesivity of, e.g., at least 10 gmf, at least 20 gmf, at least 30 gmf, at least 40 gmf, at least 50 gmf, at least 60 gmf, at least 70 gmf, at least 80 gmf, at least 90 gmf, at least 100 gmf, at least 150 gmf, or at least 200 gmf. In yet other aspects of this embodiment, a hydrogel composition comprising crosslinked glycosaminoglycan polymers as disclosed herein has a cohesivity of, e.g., at most 10 gmf, at most 20 gmf, at most 30 gmf, at most 40 gmf, at most 50 gmf, at most 60 gmf, at most 70 gmf, at most 80 gmf, at most 90 gmf, at most 100 gmf, at most 150 gmf, or at most 200 gmf. In yet other aspects of this embodiment, a hydrogel composition comprising crosslinked glycosaminoglycan polymers as disclosed herein has a cohesivity of, e.g., about 50 gmf to about 150 gmf, about 60 gmf to about 140 gmf, about 70 gmf to about 130 gmf, about 80 gmf to about 120 gmf, or about 90 gmf to about 110 gmf.

In yet other aspects of this embodiment, a hydrogel composition comprising crosslinked glycosaminoglycan polymers as disclosed herein has a cohesivity of, e.g., about 10 gmf to about 50 gmf, about 25 gmf to about 75 gmf, about 50 gmf to about 150 gmf, about 100 gmf to about 200 gmf, about 100 gmf to about 300 gmf, about 100 gmf to about 400 gmf, about 100 gmf to about 500 gmf, about 200 gmf to about 300 gmf, about 200 gmf to about 400 gmf, about 200 gmf to about 500 gmf, about 200 gmf to about 600 gmf, about 200 gmf to about 700 gmf, about 300 gmf to about 400 gmf, about 300 gmf to about 500 gmf, about 300 gmf to about 600 gmf, about 300 gmf to about 700 gmf, about 300 gmf to about 800 gmf, about 400 gmf to about 500, about 400 gmf to about 600, about 400 gmf to about 700, about 400 gmf to about 800, about 500 gmf to about 600 gmf, about 500 gmf to about 700 gmf, about 500 gmf to about 800 gmf, about 600 gmf to about 700 gmf, about 600 gmf to about 800 gmf, about 700 gmf to about 800 gmf, about 1000 gmf to about 2000 gmf, about 1000 gmf to about 3000 gmf, or about 2000 gmf to about 3000 gmf.

Aspects of the present specification provide, in part, a hydrogel composition comprising crosslinked glycosaminoglycan polymers as disclosed herein that exhibits a physiologically-acceptable osmolarity. As used herein, the term “osmolarity” refers to the concentration of osmotically active solutes in solution. As used herein, the term “a physiologically-acceptable osmolarity” refers to an osmolarity in accord with, or characteristic of, the normal functioning of a living organism. As such, administration of a hydrogel composition as disclosed herein exhibits an osmolarity that has substantially no long term or permanent detrimental effect when administered to a mammal. Osmolarity is expressed in terms of osmoles of osmotically active solute per liter of solvent (Osmol/L or Osm/L). Osmolarity is distinct from molarity because it measures moles of osmotically active solute particles rather than moles of solute. The distinction arises because some compounds can dissociate in solution, whereas others cannot. The osmolarity of a solution can be calculated from the following expression: Osmol/L=Σ φᵢ η₁ Cᵢ, where φ is the osmotic coefficient, which accounts for the degree of non-ideality of the solution; η is the number of particles (e.g. ions) into which a molecule dissociates; and C is the molar concentration of the solute; and i is the index representing the identity of a particular solute. The osmolarity of a hydrogel composition disclosed herein can be measured using a conventional method that measures solutions.

In an embodiment, a hydrogel composition comprising crosslinked glycosaminoglycan polymers as disclosed herein exhibits a physiologically-acceptable osmolarity. In aspects of this embodiment, a hydrogel composition exhibits an osmolarity of, e.g., about 100 mOsm/L, about 150 mOsm/L, about 200 mOsm/L, about 250 mOsm/L, about 300 mOsm/L, about 350 mOsm/L, about 400 mOsm/L, about 450 mOsm/L, or about 500 mOsm/L. In other aspects of this embodiment, a hydrogel composition exhibits an osmolarity of, e.g., at least 100 mOsm/L, at least 150 mOsm/L, at least 200 mOsm/L, at least 250 mOsm/L, at least 300 mOsm/L, at least 350 mOsm/L, at least 400 mOsm/L, at least 450 mOsm/L, or at least 500 mOsm/L. In yet other aspects of this embodiment, a hydrogel composition exhibits an osmolarity of, e.g., at most 100 mOsm/L, at most 150 mOsm/L, at most 200 mOsm/L, at most 250 mOsm/L, at most 300 mOsm/L, at most 350 mOsm/L, at most 400 mOsm/L, at most 450 mOsm/L, or at most 500 mOsm/L. In still other aspects of this embodiment, a hydrogel composition exhibits an osmolarity of, e.g., about 100 mOsm/L to about 500 mOsm/L, about 200 mOsm/L to about 500 mOsm/L, about 200 mOsm/L to about 400 mOsm/L, about 300 mOsm/L to about 400 mOsm/L, about 270 mOsm/L to about 390 mOsm/L, about 225 mOsm/L to about 350 mOsm/L, about 250 mOsm/L to about 325 mOsm/L, about 275 mOsm/L to about 300 mOsm/L, or about 285 mOsm/L to about 290 mOsm/L.

Aspects of the present specification provide, in part, a hydrogel composition comprising crosslinked glycosaminoglycan polymers as disclosed herein that exhibits a physiologically-acceptable osmolality. As used herein, the term “osmolality” refers to the concentration of osmotically active solutes per kilo of solvent in the body. As used herein, the term “a physiologically-acceptable osmolality” refers to an osmolality in accord with, or characteristic of, the normal functioning of a living organism. As such, administration of a hydrogel composition disclosed herein exhibits an osmolality that has substantially no long term or permanent detrimental effect when administered to a mammal. Osmolality is expressed in terms of osmoles of osmotically active solute per kilogram of solvent (osmol/kg or Osm/kg) and is equal to the sum of the molalities of all the solutes present in that solution. The osmolality of a solution can be measured using an osmometer. The most commonly used instrument in modern laboratories is a freezing point depression osmometer. This instruments measure the change in freezing point that occurs in a solution with increasing osmolality (freezing point depression osmometer) or the change in vapor pressure that occurs in a solution with increasing osmolality (vapor pressure depression osmometer).

In an embodiment, a hydrogel composition comprising crosslinked glycosaminoglycan polymers as disclosed herein exhibits a physiologically-acceptable osmolality. In aspects of this embodiment, a hydrogel composition exhibits an osmolality of, e.g., about 100 mOsm/kg, about 150 mOsm/kg, about 200 mOsm/kg, about 250 mOsm/kg, about 300 mOsm/kg, about 350 mOsm/kg, about 400 mOsm/kg, about 450 mOsm/kg, or about 500 mOsm/kg. In other aspects of this embodiment, a hydrogel composition exhibits an osmolality of, e.g., at least 100 mOsm/kg, at least 150 mOsm/kg, at least 200 mOsm/kg, at least 250 mOsm/kg, at least 300 mOsm/kg, at least 350 mOsm/kg, at least 400 mOsm/kg, at least 450 mOsm/kg, or at least 500 mOsm/kg. In yet other aspects of this embodiment, a hydrogel composition exhibits an osmolality of, e.g., at most 100 mOsm/kg, at most 150 mOsm/kg, at most 200 mOsm/kg, at most 250 mOsm/kg, at most 300 mOsm/kg, at most 350 mOsm/kg, at most 400 mOsm/kg, at most 450 mOsm/kg, or at most 500 mOsm/kg. In still other aspects of this embodiment, a hydrogel composition exhibits an osmolality of, e.g., about 100 mOsm/kg to about 500 mOsm/kg, about 200 mOsm/kg to about 500 mOsm/kg, about 200 mOsm/kg to about 400 mOsm/kg, about 300 mOsm/kg to about 400 mOsm/kg, about 270 mOsm/kg to about 390 mOsm/kg, about 225 mOsm/kg to about 350 mOsm/kg, about 250 mOsm/kg to about 325 mOsm/kg, about 275 mOsm/kg to about 300 mOsm/kg, or about 285 mOsm/kg to about 290 mOsm/kg.

Aspects of the present specification provide, in part, a hydrogel composition comprising crosslinked glycosaminoglycan polymers as disclosed herein that is a pharmaceutical composition. As used herein, the term “pharmaceutical composition” is synonymous with “pharmaceutically-acceptable composition” and refers to a therapeutically effective concentration of an active ingredient, such as, e.g., any of the glycosaminoglycan polymers disclosed herein. A pharmaceutical composition is useful for medical and veterinary applications. A pharmaceutical composition may be administered to an individual alone, or in combination with other supplementary active ingredients, agents, drugs or hormones.

Aspects of the present specification provide, in part, a hydrogel composition as disclosed herein that is a pharmaceutical composition comprising a pharmacologically acceptable excipient. As used herein, the term “pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. It is also envisioned that a pharmaceutical composition as disclosed herein can include one or more pharmaceutically acceptable excipients that facilitate processing of an active ingredient into pharmaceutically acceptable compositions. Insofar as any pharmacologically acceptable excipient is not incompatible with the active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

It is further envisioned that a pharmaceutical composition as disclosed herein may optionally include, or not include, without limitation, other pharmaceutically acceptable components (or pharmaceutical components), including, without limitation, buffers, preservatives, tonicity adjusters, salts, antioxidants, osmolality adjusting agents, emulsifying agents, wetting agents, sweetening or flavoring agents, and the like.

Pharmaceutically acceptable buffer is any buffer that can be used to prepare a pharmaceutical composition disclosed herein, provided that the resulting preparation is pharmaceutically acceptable. Non-limiting examples of pharmaceutically acceptable buffers include acetate buffers, borate buffers, citrate buffers, neutral buffered salines, phosphate buffers, and phosphate buffered salines. Any concentration of a pharmaceutically acceptable buffer can be useful in formulating a pharmaceutical composition disclosed herein, with the proviso that a therapeutically effective amount of the active ingredient is recovered using this effective concentration of buffer. Non-limiting examples of concentrations of physiologically-acceptable buffers occur within the range of about 0.1 mM to about 900 mM. The pH of pharmaceutically acceptable buffers may be adjusted, provided that the resulting preparation is pharmaceutically acceptable. It is understood that acids or bases can be used to adjust the pH of a pharmaceutical composition as needed. Any buffered pH level can be useful in formulating a pharmaceutical composition, with the proviso that a therapeutically effective amount of the matrix polymer active ingredient is recovered using this effective pH level. Non-limiting examples of physiologically-acceptable pH occur within the range of about pH 5.5 to about pH 8.5.

Pharmaceutically acceptable antioxidants include, without limitation, sodium metabisulfite, sodium thiosulfate, acetylcysteine, butylated hydroxyanisole and butylated hydroxytoluene. Pharmaceutically acceptable preservatives include, without limitation, benzalkonium chloride, chlorobutanol, thimerosal, phenylmercuric acetate, phenylmercuric nitrate, a stabilized oxy chloro composition, such as, e.g., PURITE® (Allergan, Inc. Irvine, Calif.) and chelants, such as, e.g., DTPA or DTPA-bisamide, calcium DTPA, and CaNaDTPA-bisamide.

Tonicity adjustors useful in a pharmaceutical composition disclosed herein include, without limitation, salts such as, e.g., sodium chloride and potassium chloride; and glycerin. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention. Other non-limiting examples of pharmacologically acceptable components can be found in, e.g., Ansel, supra, (1999); Gennaro, supra, (2000); Hardman, supra, (2001); and Rowe, supra, (2003), each of which is hereby incorporated by reference in its entirety.

A pharmaceutical compositions disclosed in the present specification generally is administered as a pharmaceutical acceptable composition comprising a matrix polymer active ingredient. As used herein, the term “pharmaceutically acceptable” means any molecular entity or composition that does not produce an adverse, allergic or other untoward or unwanted reaction when administered to an individual.

Aspects of the present specification provide, in part, a method of treating a soft tissue condition of an individual by administering a hydrogel composition as disclosed herein. As used herein, the term “treating,” refers to improving a soft tissue condition by reducing or eliminating in an individual a cosmetic or clinical symptom associated with the soft tissue condition; improving a soft tissue condition by delaying or preventing in an individual the onset of a cosmetic or clinical symptom associated with a soft tissue condition; or improving a soft tissue condition by providing a clinical or cosmetic benefit. The effectiveness of a hydrogel composition disclosed herein in treating a soft tissue condition can be determined by observing one or more cosmetic, clinical symptoms or measures, and/or physiological indicators associated with the condition. Those of skill in the art will know the appropriate symptoms, measures, and/or indicators associated with a specific soft tissue condition and will know how to determine if an individual is a candidate for treatment with a hydrogel composition disclosed herein.

In aspects of this embodiment, the term “treating” refers to reducing a symptom of a soft tissue condition by, e.g., at least 20%, at least 30%, at least 40%, at least 50%, at least 60%, at least 70%, at least 80%, at least 90% or at least 100%. In other aspects of this embodiment, the term “treating” refers to improving an aspect of measure of a soft tissue condition by, e.g., at least 20%, at least 30%, at least 40%, at least 50%, at least 60%, at least 70%, at least 80%, at least 90% or at least 100%. In addition, an improvement in a soft tissue condition can also be indicated by a reduced need for a concurrent therapy.

The hydrogel composition and methods disclosed herein are useful in treating a soft tissue condition. A soft tissue condition includes, without limitation, a soft tissue imperfection, defect, disease, and/or disorder. Non-limiting examples of a soft tissue condition include breast imperfection, defect, disease and/or disorder, such as, e.g., a breast augmentation, a breast reconstruction micromastia, thoracic hypoplasia, Poland's syndrome, defects due to implant complications like capsular contraction and/or rupture; a facial imperfection, defect, disease or disorder, such as, e.g., a facial augmentation, a facial reconstruction, Parry-Romberg syndrome, lupus erythematosus profundus, dermal divots, sunken cheeks, thin lips, nasal imperfections or defects, retro-orbital imperfections or defects, a facial fold, line and/or wrinkle like a glabellar line, a nasolabial line, a perioral line, and/or a marionette line, and/or other contour deformities or imperfections of the face; a neck imperfection, defect, disease or disorder; a skin imperfection, defect, disease and/or disorder; other soft tissue imperfections, defects, diseases and/or disorders, such as, e.g., an augmentation or a reconstruction of the upper arm, lower arm, hand, shoulder, back, torso including abdomen, buttocks, upper leg, lower leg including calves, foot including plantar fat pad, eye, genitals, or other body part, region or area, or a disease or disorder affecting these body parts, regions or areas; urinary incontinence, fecal incontinence, other forms of incontinence; and gastroesophageal reflux disease (GERD).

The amount of hydrogel composition used with any of the methods as disclosed herein will typically be determined based on the alteration and/or improvement desired, the reduction and/or elimination of a soft tissue condition symptom desired, the clinical and/or cosmetic effect desired by the individual and/or physician, and the body part or region being treated. The effectiveness of composition administration for treating a soft tissue condition may be manifested by one or more of the following clinical and/or cosmetic measures: altered and/or improved soft tissue shape, altered and/or improved soft tissue size, altered and/or improved soft tissue contour, altered and/or improved tissue function, sustained engraftment of composition, improved patient satisfaction and/or quality of life. For example, effectiveness of the compositions and methods in treating a facial soft tissue may be manifested by one or more of the following clinical and/or cosmetic measures: increased size, shape, and/or contour of facial feature like increased size, shape, and/or contour of lip, cheek or eye region; altered size, shape, and/or contour of facial feature like altered size, shape, and/or contour of lip, cheek or eye region shape; reduction or elimination of a wrinkle, fold or line in the skin; resistance to a wrinkle, fold or line in the skin; rehydration of the skin; increased elasticity to the skin; reduction or elimination of skin roughness; increased and/or improved skin tautness; reduction or elimination of stretch lines or marks; increased and/or improved skin tone, shine, brightness and/or radiance; increased and/or improved skin color, reduction or elimination of skin paleness; sustained engraftment of composition; decreased side effects; improved patient satisfaction and/or quality of life.

In aspects of this embodiment, the amount of a hydrogel composition administered is, e.g., about 0.01 g, about 0.05 g, about 0.1 g, about 0.5 g, about 1 g, about 5 g, about 10 g, about 20 g, about 30 g, about 40 g, about 50 g, about 60 g, about 70 g, about 80 g, about 90 g, about 100 g, about 150 g, or about 200 g. In other aspects of this embodiment, the amount of a hydrogel composition administered is, e.g., about 0.01 g to about 0.1 g, about 0.1 g to about 1 g, about 1 g to about 10 g, about 10 g to about 100 g, or about 50 g to about 200 g. In yet other aspects of this embodiment, the amount of a hydrogel composition administered is, e.g., about 0.01 mL, about 0.05 mL, about 0.1 mL, about 0.5 mL, about 1 mL, about 5 mL, about 10 mL, about 20 mL, about 30 mL, about 40 mL, about 50 mL, about 60 mL, about 70 g, about 80 mL, about 90 mL, about 100 mL, about 150 mL, or about 200 mL. In other aspects of this embodiment, the amount of a hydrogel composition administered is, e.g., about 0.01 mL to about 0.1 mL, about 0.1 mL to about 1 mL, about 1 mL to about 10 mL, about 10 mL to about 100 mL, or about 50 mL to about 200 mL.

The duration of treatment will typically be determined based on the cosmetic and/or clinical effect desired by the individual and/or physician and the body part or region being treated. In aspects of this embodiment, administration of a hydrogel composition disclosed herein can treat a soft tissue condition for, e.g., about 6 months, about 7 months, about 8 months, about 9 months, about 10 months, about 11 months, about 12 months, about 13 months, about 14 months, about 15 months, about 18 months, or about 24 months. In other aspects of this embodiment, administration of a hydrogel composition disclosed herein can treat a soft tissue condition for, e.g., at least 6 months, at least 7 months, at least 8 months, at least 9 months, at least 10 months, at least 11 months, at least 12 months, at least 13 months, at least 14 months, at least 15 months, at least 18 months, or at least 24 months. In yet aspects of this embodiment, administration of a hydrogel composition disclosed herein can treat a soft tissue condition for, e.g., about 6 months to about 12 months, about 6 months to about 15 months, about 6 months to about 18 months, about 6 months to about 21 months, about 6 months to about 24 months, about 9 months to about 12 months, about 9 months to about 15 months, about 9 months to about 18 months, about 9 months to about 21 months, about 6 months to about 24 months, about 12 months to about 15 months, about 12 months to about 18 months, about 12 months to about 21 months, about 12 months to about 24 months, about 15 months to about 18 months, about 15 months to about 21 months, about 15 months to about 24 months, about 18 months to about 21 months, about 18 months to about 24 months, or about 21 months to about 24 months.

Aspects of the present specification provide, in part, administering a hydrogel composition as disclosed herein. As used herein, the term “administering” means any delivery mechanism that provides a composition disclosed herein to an individual that potentially results in treatment of a soft tissue condition. The actual delivery mechanism used to administer a composition to an individual can be determined by a person of ordinary skill in the art by taking into account factors, including, without limitation, the type of soft tissue condition, the location of the soft tissue condition, the cause of the soft tissue condition, the severity of the soft tissue condition, the degree of relief desired, the duration of relief desired, the particular composition used, the rate of degradation of the particular composition used, the pharmacodynamics of the particular composition used, the nature of the other compounds included in the particular composition used, the particular route of administration, the particular characteristics, history and risk factors of the individual, such as, e.g., age, weight, general health and the like, or any combination thereof. A hydrogel composition disclosed herein may be administered by any means known to persons of ordinary skill in the art including, without limitation, syringe with needle, catheter, topically, or by direct surgical implantation.

The route of administration of a hydrogel composition to an individual patient will typically be determined based on the cosmetic and/or clinical effect desired by the individual and/or physician and the body part or region being treated. The composition as disclosed herein can be locally administered into, or in the vicinity of, a soft tissue condition, such as, e.g., a dermal region or a hypodermal region.

Aspects of the present specification provide, in part, a dermal region. As used herein, the term “dermal region” refers to the region of soft tissue comprising the epidermal-dermal junction and the dermis of the skin including the superficial dermis (papillary region) and the deep dermis (reticular region). The skin is composed of three primary layers: the epidermis, which provides waterproofing and serves as a barrier to infection; the dermis, which serves as a location for the appendages of skin; and the hypodermis (subcutaneous adipose layer). The epidermis contains no blood vessels, and is nourished by diffusion from the dermis. The main type of cells which make up the epidermis are keratinocytes, melanocytes, Langerhans cells and Merkels cells.

The dermis is the layer of skin beneath the epidermis that consists of connective tissue and cushions the body from stress and strain. The dermis is tightly connected to the epidermis by a basement membrane. It also harbors many Mechanoreceptor/nerve endings that provide the sense of touch and heat. It contains the hair follicles, sweat glands, sebaceous glands, apocrine glands, lymphatic vessels and blood vessels. The blood vessels in the dermis provide nourishment and waste removal from its own cells as well as from the Stratum basale of the epidermis. The dermis is structurally divided into two areas: a superficial area adjacent to the epidermis, called the papillary region, and a deep thicker area known as the reticular region.

The papillary region is composed of loose areolar connective tissue. It is named for its fingerlike projections called papillae that extend toward the epidermis. The papillae provide the dermis with a “bumpy” surface that interdigitates with the epidermis, strengthening the connection between the two layers of skin. The reticular region lies deep in the papillary region and is usually much thicker. It is composed of dense irregular connective tissue, and receives its name from the dense concentration of collagenous, elastic, and reticular fibers that weave throughout it. These protein fibers give the dermis its properties of strength, extensibility, and elasticity. Also located within the reticular region are the roots of the hair, sebaceous glands, sweat glands, receptors, nails, and blood vessels. Tattoo ink is held in the dermis. Stretch marks from pregnancy are also located in the dermis.

The hypodermis lies below the dermis. Its purpose is to attach the dermal region of the skin to underlying bone and muscle as well as supplying it with blood vessels and nerves. It consists of loose connective tissue and elastin. The main cell types are fibroblasts, macrophages and adipocytes (the hypodermis contains 50% of body fat). Fat serves as padding and insulation for the body.

In an aspect of this embodiment, a hydrogel composition as disclosed herein is administered to a soft tissue region of an individual by injection into a dermal region or a hypodermal region. In aspects of this embodiment, a hydrogel composition disclosed herein is administered to a dermal region of an individual by injection into, e.g., an epidermal-dermal junction region, a papillary region, a reticular region, or any combination thereof.

A hydrogel composition as disclosed herein is administered to an individual. An individual is typically a human being. Typically, any individual who is a candidate for a conventional procedure to treat a soft tissue condition is a candidate for a method disclosed herein. In addition, the presently disclosed hydrogel compositions and methods may apply to individuals seeking a small/moderate enlargement, shape change or contour alteration of a body part, area, and/or region, which may not be technically possible or aesthetically acceptable with existing soft tissue implant technology. Pre-procedural evaluation typically includes routine history and physical examination in addition to thorough informed consent disclosing all relevant risks and benefits of the procedure.

### EXAMPLES

The following examples illustrate representative embodiments now contemplated, but should not be construed to limit the disclosed multifunctional PEG-based crosslinking agents, hydrogel compositions comprising glycosaminoglycan polymers crosslinked using such agents, and methods of soft tissue augmentation using such hydrogel compositions.

### Example 1

### Synthesis of a Multifunctional PEG-Based Crosslinking Agent

This example illustrates how to make a multifunctional PEG-based crosslinking agent as disclosed herein from a base polyalcohol.

A multifunctional PEG-based crosslinking agent disclosed herein can be synthesized using a general scheme below. A base polyalchohol of about 200 Da to about 10,000 Da, and having the desired length and branching, is initially reacted with sodium hydride or any other reagent that can deprotonate the hydroxyl groups and then with epichlorohydrin or any other appropriate epoxide group(s). In the schematic below, a 4-arm base alcohol is shown; where n may be an integer of 0 to 60. In addition, although the general chemical schematic is illustrated with a 4-arm base polyalchohol, a similar synthesis scheme is employed to produce other multifunctional PEG-based crosslinking agents by simply using the appropriate base polyalcohol. For example, to synthesize a bifunctional PEG-based crosslinker, a 2-arm base polyalcohol is used; to synthesis a trifunctional PEG-based crosslinker, a 3-arm base polyalcohol is used; to synthesis a pentafunctional PEG-based crosslinker, a 5-arm base polyalcohol is used; to synthesis a hexafunctional PEG-based crosslinker, a 6-arm base polyalcohol is used; to synthesis a heptafunctional PEG-based crosslinker, a 7-arm base polyalcohol is used; to synthesis an octafunctional PEG-based crosslinker, a8-arm base polyalcohol is used; to synthesis a nonafunctional PEG-based crosslinker, a 9-arm base polyalcohol is used; to synthesis a decafunctional PEG-based crosslinker, a 10-arm base polyalcohol is used; etc.

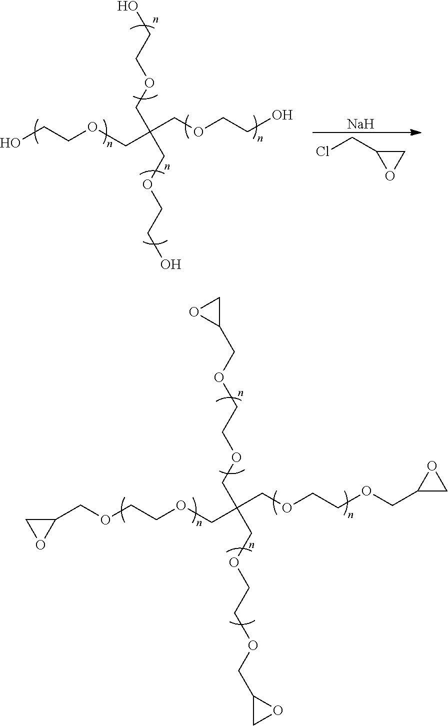

To synthesize pentaerythritol tetraglycidyl ether, 136 mg of pentaerythritol was reacted with 100 mg of sodium hydride and subsequently with 370 mg of epichlorohydrin.

### Example 2

### Crosslinking of Glycosaminoglycan Polymers Using Multifunctional PEG-Based Crosslinker

This example illustrates how to crosslink glycosaminoglycan polymers using a multifunctional PEG-based crosslinking agent as disclosed herein.

To crosslink glycosaminoglycan polymers using a multifunctional PEG-based crosslinker, 400 mg of low molecular weight sodium hyaluronate, such as, e.g., about 400,000 Da, was mixed with 2.3 grams of 1% sodium hydroxide solution and hydrated by incubating at ambient temperature for about 30 minutes. Alternatively, a high molecular weight sodium hyaluronate, such as, e.g., about 2,000,000 Da can be used. After hydration, about 80 mg (20% w/w) of a tetrafunctional PEG-based crosslinking agent of Example 1 (about 360 Da) was added to the hydrated sodium hyaluronate. The mixture was then mechanically homogenized, and then placed in an about 50° C. oven for about 90 minutes. The resulting crosslinked hydrogel is neutralized with an equimolar amount of hydrochloric acid and swelled in a phosphate buffer (pH 7.4). The resulting hydrogel comprising crosslinked hyaluronan polymers was processed once through a 60 μm mesh and dialyzed for one week using a 20 kDa MWCO bag. The dialyzed hydrogel was then transferred to 0.8 mL syringe and flash sterilized at 128° C.

A hydrogel composition as disclosed herein was alternatively produced as described above, except that after hydration, about 56 mg (14% w/w) of a tetrafunctional PEG-based crosslinking agent of Example 1 (about 360 Da) was added to the hydrated sodium hyaluronate.

To crosslink glycosaminoglycan polymers using a multifunctional PEG-based crosslinker and another non-PEG-based crosslinker, 0.4 g of sodium hyaluronate (about 400,000 Da) was mixed with 2.3 grams of 1% sodium hydroxide solution and hydrated by incubating at ambient temperature for about 30 minutes. After hydration, about 20 mg of 1,4-butanediol diglycidyl ether (BDDE) and about 80 mg of a tetrafunctional PEG-based crosslinking agent of Example 1 (about 360 Da) were added to the hydrated sodium hyaluronate. The mixture was then mechanically homogenized, and then placed in a 50° C. oven for about 90 minutes. The resulting crosslinked hydrogel is neutralized with an equimolar amount of hydrochloric acid and swelled in a phosphate buffer (pH7.4).

### Example 2A

### Crosslinking of Glycosaminoglycan Polymers Using Multifunctional PEG-Based Crosslinker

This is an example of how to make a glycosaminoglycan polymer hydrogel using a multifunctional PEG-based crosslinking agent as disclosed herein.

About 60 mg of low molecular weight sodium hyaluronate, such as, e.g., about (310000 Da-840000 Da), was hydrated in an appropriate amount of NaOH 0.25N for about 1 hour and homogenized by cartridge/cartridge mixing.

After hydration, a sufficient amount of a pentaerythrithol tetra glycidyl ether crosslinking agent (about 13% w/w) was added to the hydrated sodium hyaluronate and the mixture homogenized by cartridge/cartridge mixing and then placed in an about 50° C. oven for about 120 minutes. At this step, hydrogel had a NaHA concentration of about 135 mg/g.

The resulting crosslinked hydrogel was neutralized in a solution made of HCl 1 N/Phosphate Buffer and swollen (less than 24 H) to reach a NaHA concentration of 30 mg/g.

The resulting hydrogel comprising crosslinked hyaluronan polymers was processed once through a 100 μm mesh filter and dialyzed for about 30-50 hours against a phosphate buffer to reach a concentration of 25 mg/g in NaHA and to remove residual crosslinker. An amount of lidocaine hydrochloride was added to the hydrogel to reach a lidocaine concentration of 0.3% w/w.

The dialyzed hydrogel was then transferred to 0.8 mL COC (cyclo olefin copolymer) syringes and steam sterilized.

### Example 3

### Sizing Hydrogel Comprising Crosslinked Glycosaminoglycan Polymers

This example illustrates how to process a hydrogel composition of the invention.

To size a hydrogel comprising crosslinked glycosaminoglycan polymers, the resulting hydrogel comprising crosslinked hyaluronan polymers of Example 2 was processed once through a 60 μm mesh screen. The sized hydrogel particles were then dialyzed for one week using a 20 kDa MWCO bag. The dialyzed hydrogel was then transferred to 0.8 mL syringe and flash sterilized at 128° C.

Alternatively, the resulting hydrogel comprising crosslinked hyaluronan polymers of Example 2 was processed once through a 25 μm or a 43 μm mesh screen. The sized hydrogel particles were then dialyzed for one week using a 20 kDa MWCO bag. The dialyzed hydrogel was then transferred to 0.8 mL syringe and flash sterilized at 128° C.

To size a hydrogel comprising crosslinked glycosaminoglycan polymers, the resulting hydrogel comprising crosslinked hyaluronan polymers of Example 2 was processed five to seven times through a 105 μm mesh screen. The sized hydrogel particles were then dialyzed for one week using a 20 kDa MWCO bag. The dialyzed hydrogel was then transferred to 0.8 mL syringe and flash sterilized at 128° C.

### Example 4

### Determination of Crosslinked Glycosaminoglycan Content in Hydrogel

This example illustrates how to determine the amount of crosslinked and uncrosslinked glycosaminoglycan polymers present in a hydrogel composition.

To assess the amount of crosslinked glycosaminoglycan polymers in a hydrogel, the ratio of crosslinked and uncrosslinked glycosaminoglycan polymers present in a hydrogel was analyzed. In this assessment, the hydrogel in Example 2 that was produced using only a multifunctional PEG-based crosslinker was compared with samples of two known hyaluronan polymer-based dermal fillers: JUVÉDERM® Ultra (Allergan, Inc., Irvine Calif.) and PREVELLE™ Silk (Mentor Corp., Santa Barbara, Calif.). Test samples were briefly incubated in excess phosphor-buffered saline solution (pH 7.4) under vigorous shaking in order to leach out uncrosslinked hyaluronan polymers from the hydrogel. Crosslinked hyaluronan polymers were then removed from this solution by filtration through a 45 μm filter. The filtered solution was then analyzed using a SEC-MALS system to determine the concentration and molecular weight of the uncrosslinked hyaluronan polymers recovered.

The results indicated that hydrogels produced using a multifunctional PEG-based crosslinker as disclosed herein comprised about 26 mg/mL of hyaluronan polymers of which about 95% was crosslinked and about 5% was uncrosslinked. This analysis also showed that JUVÉDERM® Ultra comprised about 24 mg/mL of hyaluronan polymers of which about 90% was crosslinked and about 10% was uncrosslinked. In addition, this analysis showed that PREVELLE™ Silk comprised about 5 mg/mL of hyaluronan polymers of which about 99% was crosslinked and about 1% was uncrosslinked.

### Example 5

### Effects of PEG-Based Crosslinker on Hydrogel Hardness

This example illustrates how to determine the hardness or mechanical strength of a hydrogel composition.

To assess the hardness of a hydrogel, hydrogels were prepared according to the general procedure of Example 2: One was produced starting with high-molecular weight hyaluronan (about 2,000,000 Da) and using both a tetrafunctional PEG-based crosslinker (MW=5,000 Da) and BDDE. The other was produced starting with high-molecular weight hyaluronan (about 2,000,000 Da) and using BDDE as the only crosslinking agent. Both hydrogels were made in a manner that kept the molar ratio of hyaluronan polymers to crosslinker the same. The samples from the two hydrogel preparations were compared using a strain sweep test to determine gel hardness as an indicator of the degree of crosslinking of each sample. The strain sweep test was performed on an ARES rheometer using a 50 mm parallel plate set-up. Approximately 2 mL to 3 mL of each sample was placed at the center of the lower plate and the gap was set to 1 mm. The test was performed at 5 Hz frequency for a strain sweep range of 1-250%. At low values of strain, the plateau in the elastic or storage modulus G′ quantifies the gel hardness.

TABLE 2
Strain sweep test for hydrogel hardness

| BDDE | | | | | PEG-based crosslinker and BDDE | | | | |
|---|---|---|---|---|---|---|---|---|---|
| | | | | η* | | | | | η* |
| γ (%) | G′ (Pa) | G″ (Pa) | tan (δ) | (Pa · s) | γ (%) | G′ (Pa) | G″ (Pa) | tan (δ) | (Pa · s) |
| 1.04 | 77.88 | 41.73 | 0.54 | 2.81 | 1.03 | 298.81 | 51.65 | 0.17 | 9.65 |
| 1.31 | 77.75 | 42.65 | 0.55 | 2.82 | 1.30 | 297.08 | 54.79 | 0.18 | 9.62 |
| 1.66 | 77.44 | 42.68 | 0.55 | 2.81 | 1.65 | 298.54 | 55.93 | 0.19 | 9.67 |
| 2.09 | 77.54 | 42.45 | 0.55 | 2.81 | 2.06 | 298.92 | 55.41 | 0.19 | 9.68 |
| 2.62 | 77.49 | 42.53 | 0.55 | 2.81 | 2.59 | 298.34 | 55.16 | 0.18 | 9.66 |
| 3.29 | 77.46 | 42.62 | 0.55 | 2.81 | 3.26 | 297.52 | 55.40 | 0.19 | 9.63 |
| 4.12 | 77.50 | 42.70 | 0.55 | 2.82 | 4.09 | 298.07 | 55.59 | 0.19 | 9.65 |
| 5.18 | 77.55 | 42.88 | 0.55 | 2.82 | 5.15 | 297.43 | 55.37 | 0.19 | 9.63 |
| 6.50 | 77.66 | 42.95 | 0.55 | 2.82 | 6.47 | 298.32 | 55.73 | 0.19 | 9.66 |
| 8.17 | 77.68 | 42.93 | 0.55 | 2.83 | 8.13 | 297.89 | 55.76 | 0.19 | 9.65 |
| 10.25 | 77.59 | 42.91 | 0.55 | 2.82 | 10.20 | 298.12 | 55.71 | 0.19 | 9.65 |
| 12.88 | 77.41 | 42.98 | 0.56 | 2.82 | 12.81 | 297.74 | 56.05 | 0.19 | 9.64 |
| 16.18 | 77.26 | 42.93 | 0.56 | 2.81 | 16.12 | 297.45 | 56.22 | 0.19 | 9.64 |
| 20.34 | 76.96 | 43.01 | 0.56 | 2.81 | 20.22 | 296.99 | 56.39 | 0.19 | 9.62 |
| 25.53 | 76.61 | 42.98 | 0.56 | 2.80 | 25.43 | 296.40 | 56.64 | 0.19 | 9.61 |
| 32.10 | 76.06 | 43.01 | 0.57 | 2.78 | 31.95 | 294.79 | 56.89 | 0.19 | 9.56 |
| 40.29 | 75.24 | 42.99 | 0.57 | 2.76 | 40.10 | 293.53 | 57.25 | 0.20 | 9.52 |
| 50.65 | 74.11 | 42.95 | 0.58 | 2.73 | 50.45 | 291.65 | 57.68 | 0.20 | 9.46 |
| 63.68 | 72.57 | 42.93 | 0.59 | 2.68 | 63.43 | 289.30 | 58.06 | 0.20 | 9.39 |
| 80.03 | 70.52 | 42.84 | 0.61 | 2.63 | 79.73 | 286.23 | 58.50 | 0.20 | 9.30 |
| 100.60 | 67.84 | 42.64 | 0.63 | 2.55 | 100.23 | 282.42 | 58.87 | 0.21 | 9.18 |
| 126.50 | 64.48 | 42.23 | 0.65 | 2.45 | 126.06 | 277.13 | 59.24 | 0.21 | 9.02 |
| 158.93 | 60.73 | 41.65 | 0.69 | 2.34 | 158.60 | 269.19 | 59.82 | 0.22 | 8.78 |
| 200.04 | 56.63 | 40.78 | 0.72 | 2.22 | 199.26 | 258.12 | 60.74 | 0.24 | 8.44 |

Results indicated that the G′ plateau for the hyaluronan hydrogel crosslinked with a multifunctional PEG-based crosslinker and BDDE was significantly higher than that of the hydrogel crosslinked only with BDDE (Table 2).

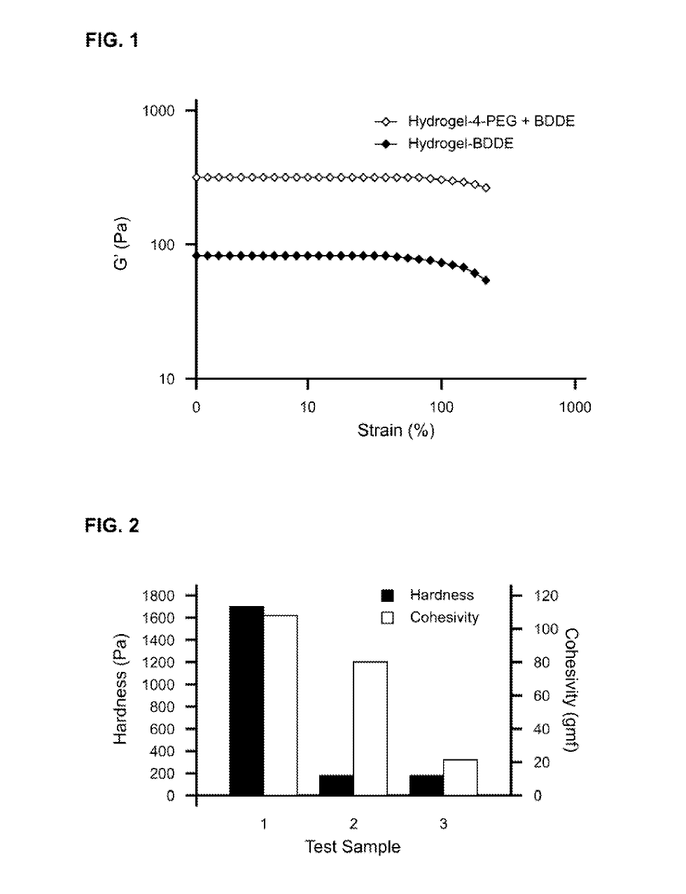

In another similar example, referring to FIG. 1, it is shown that hyaluronan hydrogel crosslinked with a multifunctional PEG-based crosslinker exhibited a G′ plateau of about 400 Pa over the range tested, whereas the hyaluronan hydrogel crosslinked only with BDDE showed a G′ plateau of about 90 Pa over a similar range.

These results indicate that the hyaluronan hydrogel crosslinked with a multifunctional PEG-based crosslinker and BDDE is harder and is more highly crosslinked than the hydrogel crosslinked only with BDDE.

### Example 6

### Effects of PEG-Based Crosslinker on Hydrogel Hardness and Cohesivity

This example illustrates how to determine the hardness or mechanical strength of a hydrogel composition as well as the cohesivity of a hydrogel composition.

To assess the cohesivity of a hydrogel, the hydrogel in Example 2 that was produced using only a multifunctional PEG-based crosslinker was compared with samples of two known hyaluronan polymer-based dermal fillers: JUVÉDERM® Ultra (Allergan, Inc., Irvine Calif.) and PREVELLE™ Silk (Mentor Corp., Santa Barbara, Calif.). The samples from the hydrogel preparations were compared using a compression test to determine gel cohesivity as an indicator of the degree of crosslinking of each sample. The cohesivity test was performed on an ARES rheometer using a 50 mm parallel plate set-up. Approximately 2 to 3 mL of each sample was placed at the center of the lower plate. The test was performed using an initial gap value of 2.5 mm, a final gap value of 0.9 mm, a compression value of 2 minutes, and a relaxation value of 12 minutes. Hydrogel hardness and cohesivity provide an indication of the lift capacity of a hydrogel, which is the ability of a hydrogel to retain its shape and resist deformation under compression.

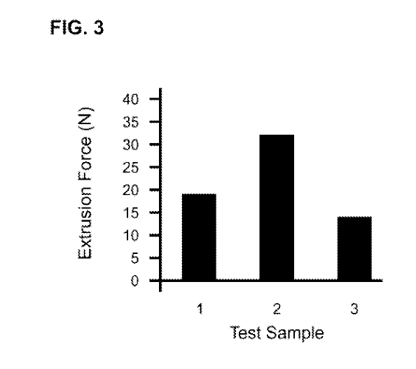

FIG. 2 summarizes the G′ plateau values at low strain as well as the maximum compression force recorded. The results indicated that the hyaluronan hydrogel crosslinked with a multifunctional PEG-based crosslinker exhibited a harness value of about 1,700 Pa and cohesivity about 95 gmf. This analysis also showed that JUVÉDERM® Ultra exhibited a harness value of about 180 Pa and cohesivity about 80 gmf. In addition, this analysis showed that PREVELLE™ Silk exhibited a harness value of about 170 Pa and cohesivity about 22 gmf.

### Example 7

### Effects of Crosslinker Ratio on Hydrogel Hardness

This example illustrates how to make a hydrogel composition using multiple crosslinking agents.

To access the effects of various ratios of two different multifunctional PEG-based crosslinkers, six samples of hydrogels comprising crosslinked hyaluronan (about 2,000,000 Da) were prepared using different ratios of bifunctional PEG-based and tetrafunctional PEG-based crosslinkers. The ratio of one bifunctional PEG-based crosslinker (MW=1,000 Da) to one tetrafunctional PEG-based crosslinker (MW=5,000 Da) was varied, such that the molar ratio of hyaluronan to total crosslinker remained the same for all six samples. The mechanical strength of each sample was tested using the same method described above in Example 4. The plateau G′ value at low strain increases as bifunctional crosslinker is replaced by equimolar amounts of the tetrafunctional crosslinker, indicating an increased degree of crosslinking (Table 3). These results indicate that as the use of tetrafunctional PEG-based crosslinkers increased the degree of crosslinking, which in turn, increased the mechanical strength of the resulting hyaluronan hydrogel.

TABLE 3
Effects of Crosslinker Ratio on Hydrogel Mechanical Strength

| Bifunctional PEG-based | Tetrafunctional PEG-based | |
|---|---|---|
| Crosslinker (1,000 Da) (%) | Crosslinker (5,000 Da) (%) | G′ (Pa) |
| 100 | 0 | 180 |
| 90 | 10 | 190 |
| 85 | 15 | 205 |
| 75 | 25 | 252 |
| 50 | 50 | 360 |
| 25 | 75 | 400 |

### Example 8

### Effects of PEG-Based Crosslinker on Hydrogel Extrusion Force

This example illustrates how to determine the extrudability of a hydrogel composition.

To assess the extrusion force necessary to inject a hydrogel through a needle, the hydrogel in Example 2 that was produced using only a multifunctional PEG-based crosslinker was compared with samples of two known hyaluronan polymer-based dermal fillers: JUVÉDERM® Ultra (Allergan, Inc., Irvine Calif.) and JUVÉDERM® Ultra Plus (Allergan, Inc., Irvine Calif.). The extrusion force test was performed by measuring the force necessary to extrude a hydrogel through a 27 gauge or 30 gauge TSK needle using a 0.8 mL Schott syringe.

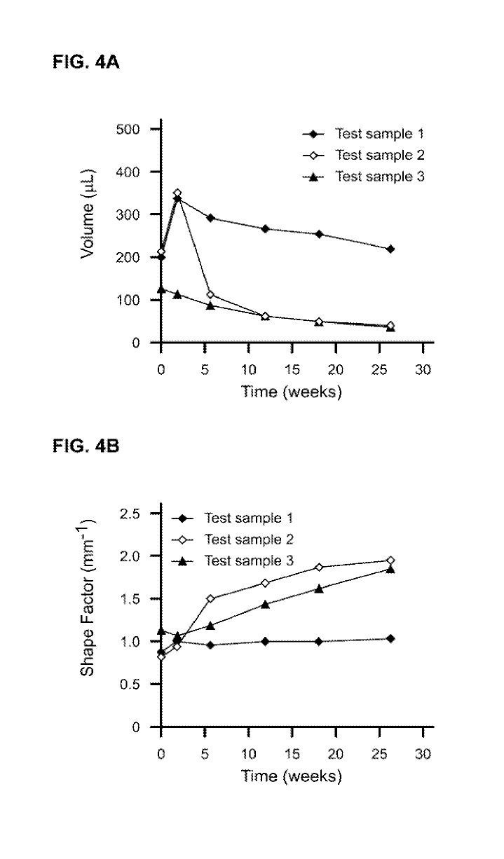

FIG. 3 summarizes the average extrusion force measured at 50 mm/minute. The results indicated that the hyaluronan hydrogel crosslinked with a multifunctional PEG-based crosslinker required about 19 N to extrude the hydrogel through a 27 gauge needle. This analysis also showed that JUVÉDERM® Ultra required about 32 N to extrude the hydrogel through a 30 gauge needle. In addition, this analysis showed that JUVÉDERM® Ultra Plus required about 14 N to extrude the hydrogel through a 27 gauge needle.

### Example 9

### Coating Hydrogel Particles with a PEG-Based Pendant Coat

This example illustrates how to coat glycosaminoglycan polymers with a PEG-based pendant group.

To coat a hydrogel comprising glycosaminoglycan polymers with a PEG-based pendant group, 380 mg of a hyaluronan-based hydrogel as disclosed herein was mixed with 20-100 mg of epoxide terminated monofunctional PEG 2000 Da and 0.5 mL of 0.01 (w/v) to 1% (w/v) sodium hydroxide. This mixture is allowed to react for 40 hours at 50° C. The resulting PEG-based pendant coated hyaluronan polymers may then be neutralized with an equimolar amount of hydrochloric acid.

Sample from hydrogels comprising PEG-based pendant coated hyaluronan polymers were compared to a sample of a hydrogels comprising non-coated hyaluronan polymers using an enzymatic degradation assay. A 0.1 mg quantity of hyaluronidase was added to the hydrogel sample and incubated for 30 minutes at 37° C. followed by 0.1 ml of a 0.8 M potassium tetraborate solution and heating at 100° C. for 10 minutes. The samples was supplemented with 3 mL of a 10% (w/v) p-dimethylaminobenzaldehyde solution in acetic acid and incubated at 37° C. for 30 minutes. The absorbance at 585 nm was used to quantify hyaluronan polymer degradation in each sample. The optical density (OD) values are reported in Table 4. As more PEG-based pendant is used to coat the hyaluronan particles, the hydrogel became less susceptible to enzymatic degradation. These results indicate that increasing the PEG-based pendant coating of glycosaminoglycan polymers of a hydrogel increases the resistance of the hydrogel to degradation.

TABLE 4
Effects of PEG-based Pendant Coating on Hyaluronan Stability

| Sample (Pendant:HA ratio) | Optical Density (OD) at 585 nm |
|---|---|
| A (0:1) | 0.750 |
| B (2:1) | 0.400 |
| C (10:1) | 0.260 |

### Example 10

### Effects of PEG-Based Crosslinker on Hydrogel Duration in vivo

This example illustrates how to determine how long a hydrogel composition will be effective in treating a soft tissue condition.

To assess the duration of a hydrogel in vivo, the hydrogel in Example 2, that was produced using only a multifunctional PEG-based crosslinker, was compared with samples of two known hyaluronan polymer-based dermal fillers: JUVÉDERM® Ultra (Allergan, Inc., Irvine Calif.) and PREVELLE™ Silk (Mentor Corp., Santa Barbara, Calif.). Female Sprague-Dawley rats (175-200 g) were injected with about 200 pL of each sample in four locations (two close to the hips and two close to the shoulders) using a 30 gauge needle. A non-evasive magnetic resonance imaging (MRI) technique was used to image the implanted grafts at six time points. The shape of the implanted hydrogel was calculated using the area to volume ratio (shape factor). A shape factor of about 0.9 mm⁻¹ corresponds to a near spherical shape of the 200 μL injection bolus, whereas a higher value corresponds to a more deformed or spread-out shape.

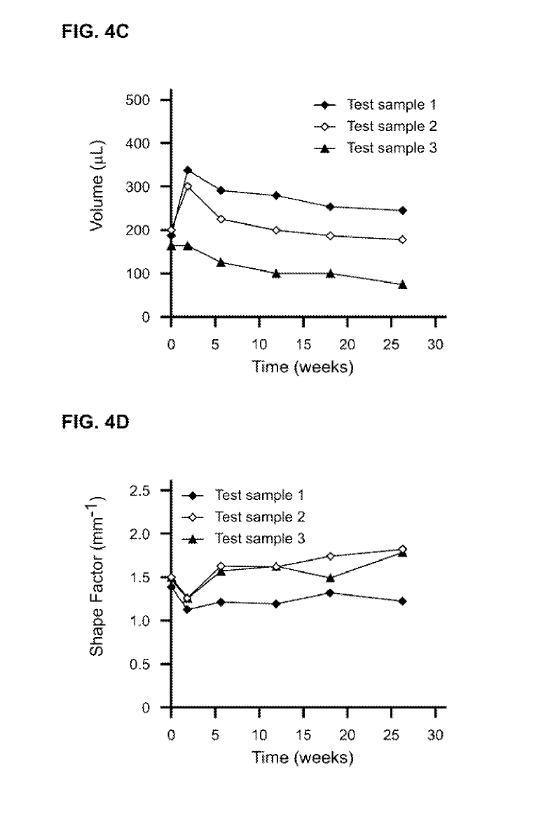

In the shoulder area, where mechanical activity is high, the hyaluronan hydrogel crosslinked with a multifunctional PEG-based crosslinker had a dramatic improvement in duration as compared to the two commercially-available hyaluronan-based dermal fillers (FIG. 4A). The hyaluronan hydrogel crosslinked with a multifunctional PEG-based crosslinker also retained a near flat spherical shape profile in the shoulder area after six months post-implantation, exhibiting a shape factor of about 1 mm⁻¹ throughout the entire time period. Both JUVÉDERM® Ultra and PREVELLE™ Silk spread significantly over the same time period, exhibiting a shape factor of about 1.25 mm⁻¹ or more after 2 months, about 1.5 mm⁻¹ or more after 4 months, and about 1.75 mm⁻¹ or more after six months (FIG. 4B).

In the hip area, where mechanical activity is low, the hyaluronan hydrogel crosslinked with a multifunctional PEG-based crosslinker also showed improvement in duration as compared to the two commercially-available hyaluronan-based dermal fillers (FIG. 4C). The hyaluronan hydrogel crosslinked with a multifunctional PEG-based crosslinker also retained a near flat spherical shape profile in the hip area after six months post-implantation, exhibiting a shape factor of about 1.25 mm⁻¹ throughout the entire time period. Both JUVÉDERM® Ultra and PREVELLE™ Silk spread significantly over the same time period, exhibiting a shape factor of about 1.25 mm⁻¹ or more after 2 months, about 1.5 mm⁻¹ or more after 4 months, and about 1.75 mm⁻¹ or more after six months (FIG. 4D).

### Example 11

### Compositions of the Invention for Treating Wrinkles

A 56-year-old, healthy male individual seeks treatment for reducing the severity of his deep nasolabial folds. His doctor injects a 0.4 cc of a hydrogel made in Example 2A just under the skin at each of the nasolabial folds.

Immediately after treatment, the individual notes that his facial expressions appear softer and more friendly. Ten months after the treatment, he reports to the doctor that he is highly satisfied with the subtle improvements in his appearance. The treatment lasts for twelve months, at which time he returns for a follow up treatment.

In closing, it is to be understood that although aspects of the present specification have been described with reference to the various embodiments, one skilled in the art will readily appreciate that the specific examples disclosed are only illustrative of the principles of the subject matter disclosed herein. Therefore, it should be understood that the disclosed subject matter is in no way limited to a particular methodology, protocol, and/or reagent, etc., described herein. As such, those skilled in the art could make numerous and various modifications or changes to or alternative configurations of the disclosed subject matter can be made in accordance with the teachings herein without departing from the spirit of the present specification. Changes in detail may be made without departing from the spirit of the invention as defined in the appended claims. Lastly, the terminology used herein is for the purpose of describing particular embodiments only, and is not intended to limit the scope of the present invention, which is defined solely by the claims. In addition, it is intended that all matter contained in the above description or shown in the accompanying drawings shall be interpreted as illustrative only and not limiting. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

What is claimed is:

1. An injectable hydrogel composition comprising:
a crosslinked hyaluronan polymer, wherein the hyaluronan polymer is crosslinked with pentaerythritol tetraglycidyl ether (PEGE) and the composition has a PEGE/hyaluranon polymer concentration of about 10% to about 15% (w/w).

2. The hydrogel composition of claim 1, wherein the hyaluronan polymer comprises low molecular weight hyaluronan.

3. The hydrogel composition of claim 1, wherein the hyaluronan polymer has a mean molecular weight of between about 310,000 Da and about 840,000 Da.

4. The hydrogel composition of claim 1, wherein the hyaluronan polymer is present in the composition at a concentration of about 18 mg/mL to about 28 mg/mL.

5. The hydrogel composition of claim 1, wherein the hyaluronan polymer is present in the composition at a concentration of about 24 mg/mL to about 26 mg/mL.

6. The hydrogel composition of claim 1, wherein the hyaluronan polymer is present in the composition at a concentration of about 25 mg/mL.

7. The hydrogel composition of claim 1 having a PEGE/hyaluron polymer concentration of about 13% (w/w).

8. The hydrogel composition of claim 1 made by the steps of
hydrating dry sodium hyaluronate (NaHA),
adding PEGE to crosslink the hydrated NaHA to obtain a PEGE/NaHA mixture.

9. A method of making an injectable hydrogel composition comprising the steps of hydrating dry sodium hyaluronate (NaHA);
adding PEGE to crosslink the hydrated NaHA to obtain a NaHA/PEGE mixture having a PEGE/NaHA concentration of about 10% to about 15% (w/w);
neutralizing and swelling the NaHA/PEGE mixture;
dialyzing the NaHA/PEGE mixture to reach a NaHA concentration of about 18 mg/mL to about 28 mg/mL.

10. The method of claim 9 wherein the NaHA/PEGE mixture has a NaHA concentration of about 135 mg/g prior to the step of neutralizing and swelling.

11. The method of claim 9 wherein the NaHA/PEGE mixture has a NaHA concentration of about 25 mg/g after the step of dialyzing.

12. A method of treating a soft tissue condition of an individual, the method comprising the steps of administering an injectable hydrogel composition made by a method of claim 9 to a site of the soft tissue condition; wherein administration of the composition improves the soft tissue condition.

13. The method of claim 12, wherein the soft tissue condition is a cosmetic defect.

14. An injectable hydrogel composition comprising sodium hyaluronate (NaHA) crosslinked with pentaerythritol tetraglycidyl ether (PEGE),
wherein the NaHA has a mean molecular weight of between about 310,000 Da and about 840,000 Da, and
wherein the NaHA is present in the composition at a concentration of about 18 mg/mL to about 28 mg/mL.

15. The injectable hydrogel composition of claim 14 wherein the hyaluronan polymer is present at a concentration of about 25 mg/mL.

16. The injectable hydrogel composition of claim 14 made by a method comprising the steps of
hydrating dry sodium hyaluronate (NaHA);
adding PEGE to crosslink the hydrated NaHA to obtain a NaHA/PEGE mixture having a PEGE/NaHA concentration of about 10% to about 15% (w/w);
neutralizing and swelling the NaHA/PEGE mixture;
dialyzing the NaHA/PEGE mixture to reach a NaHA concentration of about 18 mg/mL to about 28 mg/mL.

17. The injectable hydrogel composition of claim 16 wherein the NaHA/PEGE mixture has a NaHA concentration of about 135 mg/g prior to the step of neutralizing and swelling.

18. The injectable hydrogel composition of claim 14 wherein the NaHA is present in the composition at a concentration of about 25 mg/mL.

19. An injectable dermal filler composition for treating wrinkles in facial skin, the composition comprising a crosslinked hyaluronan polymer, wherein the hyaluronan polymer is crosslinked with pentaerythritol tetraglycidyl ether (PEGE) and the composition has a PEGE/hyaluranon polymer concentration of about 10% to about 15% (w/w).
